SEQUENCE LISTING (109 sequences)

SEQ ID NO: 1 - DNA, 21 bp, Artificial Sequence
  Description of Artificial Sequence Synthetic oligonucleotide
aaggccaagatccaagataaa

SEQ ID NO: 2 - DNA, 21 bp, Artificial Sequence
  Description of Artificial Sequence Synthetic oligonucleotide
aagaggtggtatgcagatctt

SEQ ID NO: 3 - DNA, 19 bp, Artificial Sequence
  Description of Artificial Sequence Synthetic oligonucleotide
aaatgtgaaggccaagatc

SEQ ID NO: 4 - DNA, 19 bp, Artificial Sequence
  Description of Artificial Sequence Synthetic oligonucleotide
aatgtgaaggccaagatcc

SEQ ID NO: 5 - DNA, 19 bp, Artificial Sequence
  Description of Artificial Sequence Synthetic oligonucleotide
atgtgaaggccaagatcca

SEQ ID NO: 6 - DNA, 19 bp, Artificial Sequence
  Description of Artificial Sequence Synthetic oligonucleotide
tgtgaaggccaagatccaa

SEQ ID NO: 7 - DNA, 19 bp, Artificial Sequence
  Description of Artificial Sequence Synthetic oligonucleotide
gtgaaggccaagatccaag

SEQ ID NO: 8 - DNA, 19 bp, Artificial Sequence
  Description of Artificial Sequence Synthetic oligonucleotide
tgaaggccaagatccaaga

SEQ ID NO: 9 - DNA, 19 bp, Artificial Sequence
  Description of Artificial Sequence Synthetic oligonucleotide
gaaggccaagatccaagat

SEQ ID NO: 10 - DNA, 19 bp, Artificial Sequence
  Description of Artificial Sequence Synthetic oligonucleotide
aaggccaagatccaagata

SEQ ID NO: 11 - DNA, 19 bp, Artificial Sequence
  Description of Artificial Sequence Synthetic oligonucleotide
aggccaagatccaagataa

SEQ ID NO: 12 - DNA, 19 bp, Artificial Sequence
  Description of Artificial Sequence Synthetic oligonucleotide
ggccaagatccaagataaa

SEQ ID NO: 13 - DNA, 19 bp, Artificial Sequence
  Description of Artificial Sequence Synthetic oligonucleotide
gccaagatccaagataaag

SEQ ID NO: 14 - DNA, 19 bp, Artificial Sequence
  Description of Artificial Sequence Synthetic oligonucleotide
ccaagatccaagataaaga

SEQ ID NO: 15 - DNA, 19 bp, Artificial Sequence
  Description of Artificial Sequence Synthetic oligonucleotide
caagatccaagataaagaa

SEQ ID NO: 16 - DNA, 19 bp, Artificial Sequence
  Description of Artificial Sequence Synthetic oligonucleotide
aagatccaagataaagaag

SEQ ID NO: 17 - DNA, 19 bp, Artificial Sequence
  Description of Artificial Sequence Synthetic oligonucleotide
agatccaagataaagaagg

SEQ ID NO: 18 - DNA, 19 bp, Artificial Sequence
  Description of Artificial Sequence Synthetic oligonucleotide
gatccaagataaagaaggc

SEQ ID NO: 19 - DNA, 19 bp, Artificial Sequence
  Description of Artificial Sequence Synthetic oligonucleotide
atccaagataaagaaggca

SEQ ID NO: 20 - DNA, 19 bp, Artificial Sequence
  Description of Artificial Sequence Synthetic oligonucleotide
tccaagataaagaaggcat

SEQ ID NO: 21 - DNA, 19 bp, Artificial Sequence
  Description of Artificial Sequence Synthetic oligonucleotide
ccaagataaagaaggcatc

SEQ ID NO: 22 - DNA, 21 bp, Unknown
  Description of Unknown UBB_1 sequence
caggatcctggtatccgctaa

SEQ ID NO: 23 - DNA, 21 bp, Unknown
  Description of Unknown UBB_2 sequence
atggcattactctgcactata

SEQ ID NO: 24 - DNA, 21 bp, Unknown
  Description of Unknown UBB_3 sequence
ccaacttaagtttagaaatta

SEQ ID NO: 25 - DNA, 21 bp, Unknown
  Description of Unknown UBB_4 sequence
gaggctcatctttgcaggcaa

SEQ ID NO: 26 - DNA, 19 bp, Artificial Sequence
  Description of Artificial Sequence Synthetic oligonucleotide
gaacaaccggcaaatagat

SEQ ID NO: 27 - DNA, 19 bp, Artificial Sequence
  Description of Artificial Sequence Synthetic oligonucleotide
gcaatacgcgaagacataa

SEQ ID NO: 28 - DNA, 19 bp, Artificial Sequence
  Description of Artificial Sequence Synthetic oligonucleotide
gaaagacggaccataacat

SEQ ID NO: 29 - DNA, 19 bp, Artificial Sequence
  Description of Artificial Sequence Synthetic oligonucleotide
gaagaaccacgaagactta

SEQ ID NO: 30 - DNA, 19 bp, Artificial Sequence
  Description of Artificial Sequence Synthetic oligonucleotide
gtaggacgcacaaactaaa

SEQ ID NO: 31 - DNA, 19 bp, Artificial Sequence
  Description of Artificial Sequence Synthetic oligonucleotide
ggacagatcgctaaacaaa

SEQ ID NO: 32 - DNA, 19 bp, Artificial Sequence
  Description of Artificial Sequence Synthetic oligonucleotide
ggccaagatccaggataaa

SEQ ID NO: 33 - DNA, 19 bp, Artificial Sequence
  Description of Artificial Sequence Synthetic oligonucleotide
ggccaagatccaggataag

SEQ ID NO: 34 - DNA, 19 bp, Artificial Sequence
  Description of Artificial Sequence Synthetic oligonucleotide
ggcaaagatccaagataag

SEQ ID NO: 35 - RNA, 19 nt, Artificial Sequence
  Description of Artificial Sequence Synthetic oligonucleotide
gccguacucuuucugacua

SEQ ID NO: 36 - RNA, 19 nt, Artificial Sequence
  Description of Artificial Sequence Synthetic oligonucleotide
guaugcagaucuucgugaa

SEQ ID NO: 37 - RNA, 19 nt, Artificial Sequence
  Description of Artificial Sequence Synthetic oligonucleotide
gaccaucacucuggaggug

SEQ ID NO: 38 - RNA, 19 nt, Artificial Sequence
  Description of Artificial Sequence Synthetic oligonucleotide
cccagugacaccaucgaaa

SEQ ID NO: 39 - RNA, 19 nt, Artificial Sequence
  Description of Artificial Sequence Synthetic oligonucleotide
gugaagacccugacuggua

SEQ ID NO: 40 - RNA, 19 nt, Artificial Sequence
  Description of Artificial Sequence Synthetic oligonucleotide
aagcaaagauccaggacaa

SEQ ID NO: 41 - RNA, 19 nt, Artificial Sequence
  Description of Artificial Sequence Synthetic oligonucleotide
gaagauggacgcacccugu

SEQ ID NO: 42 - RNA, 19 nt, Artificial Sequence
  Description of Artificial Sequence Synthetic oligonucleotide
guaagaccaucacucucga

SEQ ID NO: 43 - DNA, 19 bp, Artificial Sequence
  Description of Artificial Sequence Synthetic oligonucleotide
caagaccatcacccttgag

SEQ ID NO: 44 - DNA, 19 bp, Artificial Sequence
  Description of Artificial Sequence Synthetic oligonucleotide
tgcagatcttcgtgaagac

SEQ ID NO: 45 - DNA, 19 bp, Artificial Sequence
  Description of Artificial Sequence Synthetic oligonucleotide
agcccagtgacaccatcga

SEQ ID NO: 46 - DNA, 19 bp, Artificial Sequence
  Description of Artificial Sequence Synthetic oligonucleotide
gactacaacatccagaaag

SEQ ID NO: 47 - DNA, 19 bp, Artificial Sequence
  Description of Artificial Sequence Synthetic oligonucleotide
ctacaacatccagaaagag

SEQ ID NO: 48 - DNA, 19 bp, Artificial Sequence
  Description of Artificial Sequence Synthetic oligonucleotide
tgactacaacatccagaaa

SEQ ID NO: 49 - DNA, 19 bp, Artificial Sequence
  Description of Artificial Sequence Synthetic oligonucleotide
agtgacaccatcgaaaatg

SEQ ID NO: 50 - DNA, 19 bp, Artificial Sequence
  Description of Artificial Sequence Synthetic oligonucleotide
aggcaaagatccaagataa

SEQ ID NO: 51 - DNA, 19 bp, Artificial Sequence
  Description of Artificial Sequence Synthetic oligonucleotide
ggcaaagatccaagacaag

SEQ ID NO: 52 - DNA, 19 bp, Artificial Sequence
  Description of Artificial Sequence Synthetic oligonucleotide
caaggcaaagatccaagac

SEQ ID NO: 53 - DNA, 19 bp, Artificial Sequence
  Description of Artificial Sequence Synthetic oligonucleotide
aggcaaagatccaagacaa

SEQ ID NO: 54 - DNA, 19 bp, Artificial Sequence
  Description of Artificial Sequence Synthetic oligonucleotide
caggataaggaaggcattc

SEQ ID NO: 55 - DNA, 19 bp, Artificial Sequence
  Description of Artificial Sequence Synthetic oligonucleotide
caggacaaggaaggcattc

SEQ ID NO: 56 - DNA, 19 bp, Artificial Sequence
  Description of Artificial Sequence Synthetic oligonucleotide
ggcaagcagctggaagatg

SEQ ID NO: 57 - DNA, 19 bp, Artificial Sequence
  Description of Artificial Sequence Synthetic oligonucleotide
ggaaagcagctggaagatg

SEQ ID NO: 58 - DNA, 19 bp, Artificial Sequence
  Description of Artificial Sequence Synthetic oligonucleotide
gactacaacatccagaagg

SEQ ID NO: 59 - DNA, 19 bp, Artificial Sequence
  Description of Artificial Sequence Synthetic oligonucleotide
tgactacaacatccagaag

SEQ ID NO: 60 - RNA, 19 nt, Unknown
  Description of Unknown U52 sequence
ggcaaagauccaggacaag

SEQ ID NO: 61 - RNA, 25 nt, Artificial Sequence
  Description of Artificial Sequence Synthetic oligonucleotide
ggccaagauccaagauaaagaaggc

SEQ ID NO: 62 - DNA, 37 bp, Artificial Sequence
  Description of Artificial Sequence Synthetic oligonucleotide
taatacgactcactatagcgactggttacccggtcgt

SEQ ID NO: 63 - DNA, 28 bp, Artificial Sequence
  Description of Artificial Sequence Synthetic oligonucleotide
taatacgactcactatagcgactggtta

SEQ ID NO: 64 - DNA, 20 bp, Artificial Sequence
  Description of Artificial Sequence Synthetic oligonucleotide
acgaccgggtaaccagtcgc

SEQ ID NO: 65 - DNA, 64 bp, Artificial Sequence
  Description of Artificial Sequence Synthetic oligonucleotide
gtaatacgactcactataggcgactggttacccggtcgcaattggccaagatccaagataaatt

SEQ ID NO: 66 - DNA, 64 bp, Artificial Sequence
  Description of Artificial Sequence Synthetic oligonucleotide
  Description of Combined DNA/RNA Molecule Synthetic oligonucleotide
aatttatcttggaucttggccaattgcgaccgggtaaccagtcgcctatagtgagtcgtattac

SEQ ID NO: 67 - DNA, 64 bp, Artificial Sequence
  Description of Artificial Sequence Synthetic oligonucleotide
gtaatacgactcactataggcgactggttacccggtcgcaaaatttatcttggatcttggcctt

SEQ ID NO: 68 - DNA, 64 bp, Artificial Sequence
  Description of Artificial Sequence Synthetic oligonucleotide
aaggccaagatccaagataaattttgcgaccgggtaaccagtcgcctatagtgagtcgtattac

SEQ ID NO: 69 - RNA, 19 nt, Unknown
  Description of Unknown siRNA targeting sequence
ggcaaagauccaagauaag

SEQ ID NO: 70 - RNA, 19 nt, Unknown
  Description of Unknown siRNA targeting sequence
ggccaagauccaagauaaa

SEQ ID NO: 71 - RNA, 19 nt, Unknown
  Description of Unknown siRNA targeting sequence
aggccaagauccaagauaa

SEQ ID NO: 72 - DNA, 19 bp, Homo sapiens
ggccaagatccaagataaa

SEQ ID NO: 73 - DNA, 19 bp, Homo sapiens
ggccaagatccaggataaa

SEQ ID NO: 74 - DNA, 18 bp, Artificial Sequence
  Description of Artificial Sequence Synthetic oligonucleotide
ggccaagatccaagataa

SEQ ID NO: 75 - DNA, 18 bp, Homo sapiens
ggccaagatccaggataa

SEQ ID NO: 76 - DNA, 21 bp, Artificial Sequence
  Description of Artificial Sequence Synthetic oligonucleotide
  Description of Combined DNA/RNA Molecule Synthetic oligonucleotide
ggccaagauccaagauaaatt

SEQ ID NO: 77 - DNA, 21 bp, Artificial Sequence
  Description of Artificial Sequence Synthetic oligonucleotide
  Description of Combined DNA/RNA Molecule Synthetic oligonucleotide
uuuaucuuggaucuuggcctt

SEQ ID NO: 78 - DNA, 14 bp, Artificial Sequence
  Description of Artificial Sequence Synthetic oligonucleotide
aagatccaagataa

SEQ ID NO: 79 - DNA, 14 bp, Homo sapiens
aagatccaagataa

SEQ ID NO: 80 - DNA, 15 bp, Artificial Sequence
  Description of Artificial Sequence Synthetic oligonucleotide
aagatccaagataaa

SEQ ID NO: 81 - DNA, 15 bp, Homo sapiens
aagatccaagataaa

SEQ ID NO: 82 - DNA, 15 bp, Artificial Sequence
  Description of Artificial Sequence Synthetic oligonucleotide
ggccaagatccaaga

SEQ ID NO: 83 - DNA, 15 bp, Homo sapiens
ggccaagatccaaga

SEQ ID NO: 84 - DNA, 15 bp, Artificial Sequence
  Description of Artificial Sequence Synthetic oligonucleotide
gccaagatccaagat

SEQ ID NO: 85 - DNA, 15 bp, Homo sapiens
gccaagatccaagat

SEQ ID NO: 86 - DNA, 933 bp, Homo sapiens
atttaggggcggttggctttgttgggtgagcttgtttgtgtccctgtgggtggacgtggttggtgattggcaggatcctggtatccgctaacaggtcaaaatgcagatcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggtcctgcgtctgagaggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtggctgttaattcttcagtcatggcattcgcagtgcccagtgatggcattactctgcactatagccatttgccccaacttaagtttagaaattacaagtttcagtaatagctgaacctgttcaaaatgttaataaaggtttcgttgcatggta

SEQ ID NO: 87 - DNA, 2193 bp, Homo sapiens
agttccgtcgcagccgggatttgggtcgcagttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaa

SEQ ID NO: 88 - RNA, 19 nt, Artificial Sequence
  Description of Artificial Sequence Synthetic oligonucleotide
gcgacugguuacccggucg

SEQ ID NO: 89 - RNA, 45 nt, Artificial Sequence
  Description of Artificial Sequence Synthetic oligonucleotide
gcgacugguuacccggucguaaaaggccaagauccaagauaaauu

SEQ ID NO: 90 - RNA, 45 nt, Artificial Sequence
  Description of Artificial Sequence Synthetic oligonucleotide
gcgacugguuacccggucguaaaauuuaucuuggaucuuggccuu

SEQ ID NO: 91 - DNA, 240 bp, Homo sapiens
atcttcgtgaaaacccttaccggcaagaccatcacccttgaggtggagcccagtgacaccatcgaaaatgtgaaggccaagatccaggataaggaaggcattccccccgaccagcagaggctcatctttgcaggcaagcagctggaagatggccgtactctttctgactacaacatccagaaggagtcgaccctgcacctggtcctgcgtctgagaggtggtatgcagatcttcgtgaag

SEQ ID NO: 92 - DNA, 240 bp, Homo sapiens
atcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaag

SEQ ID NO: 93 - DNA, 251 bp, Homo sapiens
aaggagtcgaccctgcacctggtcctgcgtctgagaggtggtatgcagatcttcgtgaagaccctgaccggcaagaccatcaccctggaagtggagcccagtgacaccatcgaaaatgtgaaggccaagatccaggataaagaaggcatccctcccgaccagcagaggctcatctttgcaggcaagcagctggaagatggccgcactctttctgactacaacatccagaaggagtcgaccctgcacctggt

SEQ ID NO: 94 - DNA, 300 bp, Homo sapiens
aaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgcccaagacaaggaaggcattcctcctgaccagcagaggttgatctttgccggaaagcagctggaagatgggcgcaccctgtctgactacaacatccagaaagagtctaccctgcacctggtg

SEQ ID NO: 95 - DNA, 233 bp, Homo sapiens
ggtggtatgcagatcttcgtgaagaccctgaccggcaagaccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgcgcctgaggggtgg

SEQ ID NO: 96 - DNA, 240 bp, Homo sapiens
ggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcag

SEQ ID NO: 97 - DNA, 180 bp, Homo sapiens
ccatcactctggaggtggagcccagtgacaccatcgaaaatgtgaaggccaagatccaagataaagaaggcatcccccccgaccagcagaggctcatctttgcaggcaagcagctggaagatggccgcactctttctgactacaacatccagaaagagtcgaccctgcacctggtcctgc

SEQ ID NO: 98 - DNA, 180 bp, Mus musculus
ccatcaccttggaggtggagcccagtgacaccatcgagaatgtgaaggccaagatccaggataaagagggcatcccccctgaccagcagaggctgatctttgccggcaagcagctagaagatggccgcactctctctgattacaacatccagaaagagtcgaccctgcacctggtcctcc

SEQ ID NO: 99 - DNA, 180 bp, Homo sapiens
ccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctcc

SEQ ID NO: 100 - DNA, 180 bp, Mus musculus
ccatcaccctggaggtcgagcccagtgacaccattgagaatgtcaaggcaaagatccaggacaaggagggcatcccccctgaccagcagaggctgatctttgcaggcaagcagctggaagatggccgcaccctgtcagactacaacatccagaaagagtccaccctgcacctggtccttc

SEQ ID NO: 101 - DNA, 19 bp, Mus musculus
aggccaagatccaggataa

SEQ ID NO: 102 - DNA, 19 bp, Mus musculus
ggcaaagatccaggacaag

SEQ ID NO: 103 - DNA, 19 bp, Mus musculus
aggcaaagatccaggacaa

SEQ ID NO: 104 - RNA, 21 nt, Artificial Sequence
  Description of Artificial Sequence Synthetic oligonucleotide
aggccaagauccaagauaauu

SEQ ID NO: 105 - RNA, 21 nt, Artificial Sequence
  Description of Artificial Sequence Synthetic oligonucleotide
uuaucuuggaucuuggccuuu

SEQ ID NO: 106 - RNA, 27 nt, Artificial Sequence
  Description of Artificial Sequence Synthetic oligonucleotide
ggccaagauccaagauaaagaaggcuu

SEQ ID NO: 107 - RNA, 27 nt, Artificial Sequence
  Description of Artificial Sequence Synthetic oligonucleotide
gccuucuuuaucuuggaucuuggccuu

SEQ ID NO: 108 - RNA, 45 nt, Artificial Sequence
  Description of Artificial Sequence Synthetic oligonucleotide
gcgacugguuacccggucgcaaaauuuaucuuggaucuuggccuu

SEQ ID NO: 109 - RNA, 45 nt, Artificial Sequence
  Description of Artificial Sequence Synthetic oligonucleotide
gcgacugguuacccggucgcaauuggccaagauccaagauaaauu

(19) United States
(12) Patent Application Publication
(10) Pub. No.: US 2024/0229033 A1
(43) Pub. Date: Jul. 11, 2024

(54) Compositions for Inhibiting Growth of Targeted Cells

(71) Applicant: SYSTEMS ONCOLOGY, LLC, Scottsdale, AZ (US)

(72) Inventors: Spyro Mousses, Scotsdale, AZ (US); David Azorsa, Scotsdale, AZ (US); Daniel Feldheim, Boulder, CO (US); James Heil, Boulder, CO (US); Necky Tran, London, Ontario (CA); Gregory Allen Penner, Toronto, Ontario (CA)

(21) Appl. No.: 18/558,951

(22) PCT Filed: May 5, 2022
(86) PCT No.: PCT/US2022/027925

§ 371 Date: Nov. 3, 2023

Related U.S. Application Data
(60) Provisional application No. 63/185,359, filed on May 6, 2021.
(60) Provisional application No. 63/231,234, filed on Aug. 9, 2021.
(60) Provisional application No. 63/242,865, filed on Sep. 10, 2021.
(60) Provisional application No. 63/250,548, filed on Sep. 30, 2021.
(60) Provisional application No. 63/287,037, filed on Dec. 7, 2021.
(60) Provisional application No. 63/287,040, filed on Dec. 7, 2021.
(60) Provisional application No. 63/323,997, filed on Mar. 25, 2022.

(51) Int. Cl. C12N 15/113; A61P 35/00; C12N 15/115

(52) U.S. Cl. CPC C12N 15/113; A61P 35/00; C12N 15/115; C12N 2310/14; C12N 2310/16; C12N 2310/3513; C12N 2310/3515; C12N 2310/3519

(57) ABSTRACT

The invention provides siRNA compositions for inhibiting gene expression in targeted cells.

### FIELD OF THE INVENTION

The present invention is directed to therapeutic compounds and more specifically to conjugates of targeting moieties and toxins, to compositions including the same, and to methods for using the same to treat cancer, autoimmune diseases or infectious diseases. The present invention is also directed to ligand-cytotoxin conjugates, compositions and methods for using the same to treat cancer, an autoimmune disease or an infectious disease. The invention also relates to methods of using targeting molecule-cytotoxin conjugate compounds for in vitro, in situ, and in vivo diagnosis or treatment of mammalian cells, or associated pathological conditions.

### RELATED AND PRIORITY APPLICATIONS

This application is a National Stage of International Application No. PCT/US2022/027925, filed May 5, 2022, which claims priority to U.S. Provisional Patent Application No. 63/185,359 filed May 6, 2021, U.S. Provisional Patent Application No. 63/231,234 filed Aug. 9, 2021, U.S. Provisional Patent Application No. 63/242,865 filed Sep. 10, 2021, U.S. Provisional Patent Application No. 63/250,548 filed Sep. 30, 2021, U.S. Provisional Patent Application No. 63/287,037 filed Dec. 7, 2021, U.S. Provisional Patent Application No. 63/287,040 filed Dec. 7, 2021 and U.S. Provisional Patent Application No. 63/323,997 filed Mar. 25, 2022. All of the above applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on May 20, 2022, is named 10911_011097-WO0_SL.txt and is 30,629 bytes in size.

### BACKGROUND OF THE INVENTION

Traditional cancer therapy often involves a low therapeutic window and non-specific chemotherapeutic agents that also affect normal cells that have high mitotic rates. Such therapies often cause a variety of adverse effects, and in some cases lead to drug resistance. Monoclonal antibodies (mAbs) have demonstrated therapeutical for the treatment of a numerous diseases, especially cancer. The benefits of mAbs include their target specificity, wide therapeutic index, and their association with fewer side effects compared to conventional therapies, such as chemotherapy, radiation therapy and surgery.

Antibody-drug conjugates (ADCs) may provide a synergistic effect by the conjugation of a mAb to a cytotoxic drug, compared to the mAb used alone. Conjugation is an approach that enables the attachment of highly toxic drugs to a tumor specific mAb, in order to construct an ADC. As used herein, the term “drug” generally means a highly cytotoxic moiety that can be used in such conjugates (also, the “payload” or the “cytotoxin”), unless the context suggests a modified definition. An ADC is usually comprised of an mAb, a linker and a cytotoxic payload. The linker conjugates the payload to the mAb, which binds to the target that is generally overexpressed on the tumor cell, and the payload creates the primary therapeutic action. ADC payloads should be stable in storage and in the blood stream as well as have non-immunogenic effects. The main characteristics of ADCs include a good internalization rate, low immunogenicity, high specificity and affinity, a potent payload, and a stable linker.

Currently, over 100 ADCs are undergoing clinical trials, but approximately 20% have been terminated or withdrawn during either phase I or phase II. Failure has usually resulted from dose limiting toxicities.

As of 2021, there have been nine ADCs approved, including Brentuximab vedotin (Adcetris®: Seattle Genetics), Inotuzumab ozogamicin (Besponsa®: Pfizer), and trastuzumab emtansine (Kadcyla®: Genentech) approved for breast cancer. These ADCs target the CD30 receptor, CD22 receptor and HER2 (human epidermal growth factor receptor 2) receptor, respectively. Five other ADCs have been approved between 2019-2021, including Polatuzumab vedotin (Polivy®: Genentech/Roche) which targets CD79b indicated for relapsed or refractory diffuse large B-cell lymphoma, Sacituzumab govitecan (IMMU-132) (Trodelvy®: Gilead Sciences) which targets TROP-2 for the treatment of triple negative breast cancer, (fam)-trastuzumab deruxtecan (Enhertu®:Daiichi Sankyo/AstraZeneca) which is indicated for HER2+ metastatic breast cancer and targets HER2, Enfortumab vedotin (Padcev®: Astellas Pharma/Seattle Genetics) which targets nectin 4 for the treatment of urothelial cancer, and Belantamab mafodotin (Blenrep®: GlaxoSmithKline) which targets BMCA and is indicated for multiple myeloma.

Orally and parenterally delivered drugs are generally not targeted, resulting in systemic delivery of the agent to cells and tissues of the body where it is unnecessary, and often undesirable. This lack of targeting may result in adverse drug side effects, and often limits the dose of a drug that can be administered. Bioavailability and residence of oral drugs in the gut lead to additional exposure of the gut to the drug and hence risk of gut toxicities. Thus, in order to avoid the physiological effects of inappropriate delivery of agents to other cells and tissues a major goal of drug research has been to develop methods for targeting therapeutics to cells and tissues. Intracellular targeting may be achieved by methods, compounds and formulations which allow accumulation or retention of biologically active agents, i.e., active metabolites, inside cells.

mAb therapy has been established for the treatment of cancer, immunological and angiogenic disorders. The use of ADCs for the local delivery of cytotoxic or cytostatic agents, e.g., drugs to kill or inhibit tumor cells in the treatment of cancer (Syrigos and Epenetos (1999) Anticancer Research 19:605-614; Niculescu-Duvaz and Springer (1997) Adv. Drg. Del. Rev. 26:151-172; U.S. Pat. No. 4,975,278) may allow targeted delivery of the drug moiety to tumors, and intracellular accumulation therein, while systemic administration of these unconjugated drug agents may result in unacceptable levels of toxicity to normal cells as well as the tumor cells (Baldwin et al., 1986, Lancet pp. (Mar. 15, 1986):603-05; Thorpe, 1985, “antibody Carriers Of Cytotoxic Agents In Cancer Therapy: A Review,” in Monoclonal Antibodies '84: Biological And Clinical Applications, A. Pinchera et al. (eds.), pp. 475-506). Both polyclonal antibodies and monoclonal antibodies have been reported as useful in these strategies (Rowland et al., 1986, Cancer Immunol. Immunother. 21:183-87).

The primary function of the antibody component of an ADC is to bind to its selected antigen moiety. These targets are currently focused around molecules that are overexpressed or preferentially expressed by tumor cells. The antibody binding to its selected antigen on the tumor cell surface must also initiate the internalization of the entire surface complex to allow for intracellular delivery of the cytotoxic payload. This is different from more traditional therapeutic monoclonal antibodies as the antibody itself is not required to have functional activity of its own (such as initiating ADCC).

The majority of current FDA-approved ADCs are designed to target an antigen overexpressed on tumor cells. However, to increase the effectiveness of a single ADC agent among several cancer types, an emerging area of interest is targeting antigens of the tumor stroma.

Unlike therapeutic mAbs, ADCs are conjugated with highly toxic compounds. The potency of these compounds generally preclude their use as an intravenously administered therapy due to the risk of toxicities. However, only a small amount of the drug needs to be delivered to the interior of a tumor to result in efficacious effects. All of the clinically approved ADCs and the most of ADCs in development carry cytotoxic payloads such as anti-mitotic agents or DNA-binding agents.

Toxins used in antibody-toxin conjugates have included bacterial toxins such as diphtheria toxin, plant toxins such as ricin, small molecule toxins such as geldanamycin. However, some cytotoxic drugs tend to be less active when conjugated to large antibodies or protein receptor ligands. These and other limitations and problems are addressed by certain embodiments of the present invention.

Despite promising advances with ADCs, significant general toxicities at doses required for achieving a therapeutic effect compromise their efficacy in clinical studies. Accordingly, there is a clear need in the art for new cytotoxic drugs having useful therapeutic efficiency but significantly lower non-specific toxicity. These and other limitations and problems are addressed by certain embodiments of the present invention.

Chemical linkers are used to attach a cytotoxic payload to a mAb and derive the efficient delivery of the payload, but the linker is also a determinant of the toxicity of an ADC. Linkers are comprised of various functional groups in order to conjugate the cytotoxic payload to the mAb as well as to control the distribution and release of the payload into the targeted cell. The release of the payload from the antibody can cause potent off-target toxicities if, for example, the toxic payload is released before entering the tumor.

The chemical linkers used currently are either cleavable or non-cleavable.

Cleavable linkers are characterized by a cleavage site located between the cytotoxic payload and the mAb. This cleavage can occur by physiological stimuli and by multiple different mechanism based on the specific linker chemistry: acid-labile hydrolysis, enzymatic, or reductive for example.

Non-cleavable linkers are meant to be stable and prevent the release of the payload while in circulation or in extravascular spaces and only releases once it is internalized.

The use of non-cleavable linkers on an ADC may provide an improved therapeutic index due to the improved plasma stability, however, certain cleavable linker chemistries have been developed that improve plasma stability. There are hundreds of linkers known in the art and in development which can be adapted for use in the present invention by one skilled in the art.

Cytotoxin-related toxicities are more evident in animal studies when no binding to the target occurred than when the ADCs bound to their targets (Saber et al., Regul. Toxicol. Pharmacol. 2015). Freely circulating cytotoxins create high non-specific toxicity. Fast growing cells (e.g., bone marrow, intestinal mucosa, and the hair follicle cells) are most affected. Such toxicity limits the therapeutic for these otherwise effective drugs and creates a need in the art for new cytotoxic payloads as provided by embodiments of the instant invention.

RNA interference (RNAi), also known as RNA silencing, has been extensively explored for therapeutic use in reducing gene expression but in the decades since its discovery few therapeutics have been approved. The primary hurdle for clinical advancement of siRNAs is the delivery. SIRNA's have poor cellular uptake and unfavorable pharmacokinetics, including nuclease degradation and rapid clearance from the systematic circulation (Charbe et al., Acta Pharm Sin B., 2020; 10(11): 2075-2109). For these reasons siRNA related toxicity is very low. The nucleic acid compounds and methods of using the same as provided herein solve these and other problems in the art.

A number of nonviral delivery carriers, including liposomes, lipids, polymers, peptides, virus-based vectors, and pressurized hydrodynamic injection, are being researched for improved intracellular delivery of siRNA. In addition, various kinds of cationic species can form nano-sized complexes with negatively charged siRNA by ionic interactions. The resulting complexes can provide protection of siRNA and allow cellular uptake.

It has been reported that direct conjugation of small drug molecules, aptamers, lipids, peptides, proteins, or polymers to siRNA can improve in vivo pharmacokinetic behavior of siRNA. Such siRNA conjugates, either with or without forming nano-complexes with cationic carriers, could enhance biological half-life with an increase of delivery efficiency to the target tissue.

U.S. Pat. No. 10,426,842B2 discloses a nanoparticle including an siRNA and a cytotoxin. This patent teaches that standalone siRNA with targeted delivery does not cause cytotoxicity to cells to the extent that infected cells are eliminated. A knockdown of an oncogene merely silences the gene of interest, and consequently facilitates the affected cancer cells to adapt and adopt a different pathway of survival.

EP Patent No EP2164868A1 discloses a polypeptide comprising a therapeutic agent which is either a cytotoxin or an siRNA.

Dual targeting of genes by a single siRNA through targeting conserved homologous regions has been shown to be effective to inhibit the expression of gene families by diminishing the function of escape pathways. In vitro, a multi-target siRNA targeting the conserved homology region of DNMT3 family members effectively inhibited expression (Du et al., Gen and Mol Bio, 35:164-171(2012)).

Recent work has expanded the RNA constructs to include joining two siRNAs to inhibit two different targets (Liu et al., Sci Reports, 6: (2016)). SiRNA's processed by cellular RNAi machinery to produce two siRNAs as opposed to dual administration offers a number of benefits including increased circulating half-life and reduced renal excretion (Liu et al., Sci Reports, 6: (2016)).

U.S. Pat. No. 10,689,654 discloses a bivalent siRNA chimera capable of silencing two or more genes. Methods of using the bivalent siRNA chimeras for selectively targeting cells to down-regulate the expression of multiple genes is also disclosed

U.S. Pat. No. 9,953,131 discloses a method for designing a dual-targeting short interfering RNAs (siRNAs) in which both strands are deliberately designed to separately target different mRNA transcripts with complete complementarity.

U.S. Pat. No. 9,695,425 discloses an siRNA molecule that, when internalized by a B cell, suppresses expression of BAFF-R and one other target oncogene selected from: Bcl6, Bcl2, STAT3, Cyclin D1, Cyclin E2 and c-myc.

U.S. Pat. No. 10,689,654 discloses a bivalent siRNA chimera capable of silencing two or more genes. Methods of using the bivalent siRNA chimeras for selectively targeting cells to down-regulate the expression of multiple genes is also disclosed

Du et al., Gen and Mol Bio, 35:164-171(2012) discloses a siRNA targeting the conserved homologous region of DNMT3 family members.

Tsai et al., 2011, J Neurooncol. 103(2): 255-266, describes a bispecific ligand-directed toxin (truncated pseudomonas exotoxin (PE38)) designed to simultaneously target epidermal growth factor receptor (EGFR) on solid tumors and urokinase receptor (uPAR) on tumor neovasculature.

U.S. Pat. No. 7,947,289 discloses compositions comprising modified bacterial toxins and methods for using the modified bacterial toxins for targeting particular cell populations and for treating diseases.

U.S. Pat. Application No. US20040249130A1 discloses an aptamer-conjugate therapeutic agent comprising a targeting moiety conjugated to a cytotoxic moiety.

U.S. Pat. Application No. US20190225711A1 discloses constructs comprising peptides capable of targeting at least two different extracellular tumor antigens and a toxin for the treatment of cancer.

Metz, S., et al. (Proc. Natl. Acad. Sci. USA 108 (2011) 8194-8424) reports bispecific digoxigenin-binding antibodies for targeted payload delivery.

U.S. Pat. No. 10,519,249 reports a conjugate of a haptenylated polypeptide toxin and an anti-hapten antibody.

Delivery to tissues other than the liver has remained a complication and hinderance for RNAi therapies. Aptamer-siRNA chimeras have been used to effectively deliver siRNAs to downregulate expression of oncological genes targets (Liu et al., Sci Reports, 6: (2016)).

U.S. Pat. No. 10,689,654 discloses a bivalent siRNA chimera platform that incorporates two aptamers for increase efficiency of delivering siRNAs to the targeted cell. Furthermore, those aptamers are conjugated to an siRNA construct that is processed by cellular RNAi machinery to produce at least two different siRNAs to inhibit expression of two or more different genes.

U.S Pat Application US 2020-0157542 discloses a bispecific aptamer.

U.S. Pat. No. 9,567,586 discloses an EPCAM aptamer coupled to an siRNA.

U.S. Pat. No. 10,385,343 discloses a method of treating cancer by administering a chimeric molecule comprising an EPCAM binding aptamer domain and an inhibitory nucleic acid domain that targets Plk1.

Patent Application PCT/US2020/038355 discloses an EpCAM-binding aptamer domain conjugated to an siRNA that inhibits the expression of a gene selected from the group consisting of: UPF2; PARP1; APE1; PD-L1; MCL1; PTPN2; SMG1; TREX1; CMAS; and CD47 for the purpose of treating cancer.

U.S. Pat. No. 10,960,086 discloses an siRNA-aptamer chimera that utilizes two aptamers targeting HER2 and HER3 and an siRNA targeting EGFR.

U.S. Pat. No. 8,828,956 discloses a conjugate delivery platform utilizing N-acetylgalactosamine (GalNAc)-siRNA conjugates that enables subcutaneous dosing of RNAi therapeutics with potent and durable effects and a wide therapeutic index. This delivery system is only effective for delivering to the liver as GalNAc binds to the Asialoglycoprotein receptor (ASGPR) that is predominantly expressed on liver hepatocytes.

U.S. Pat. No. 8,058,069 discloses lipid nanoparticle (LNP) delivery technology. LNP technology (formerly referred to as stable nucleic acid-lipid particles or SNALP) encapsulates siRNAs with high efficiency in uniform lipid nanoparticles that are claimed to be effective in delivering RNAi therapeutics to disease sites in various preclinical models.

U.S. Pat. No. 10,278,986 discloses an antibody conjugated to an siRNA as a delivery mechanism. The antibody targets C5aR and the siRNA targets C5 expression for the treatment of rheumatoid arthritis.

Patent Application PCT/US2020/036307 discloses a method of preparing an antibody covalently linked to one or more oligonucleotides.

Many delivery methods are in development to expand tissue targeting, efficacy, and stability. Additionally, may different methods of linking the delivery agent to RNAi therapies have been explored.

Aptamer-siRNA chimeras have been used to effectively deliver siRNAs to downregulate expression of oncological genes targets (Liu et al., Sci Reports, 6: (2016)).

U.S. Pat. No. 8,685,937 the Giangrande group discloses optimizations of their original PSMA AsiC (A10-Plk1) to increase targeting specificity and silencing potency of the RNA drug. This was the first demonstration of efficacy upon systemic administration of an AsiC.

PCT Application PCT/US2011/032385 discloses an aptamer-siRNA chimera tethered via a linker of between 2-10 Uracils.

Zhou et al. (2008), Molecular Therapy, describes an aptamer covalently appended to the sense strand of an siRNA with a 4-nt linker (CUCU) between the siRNA and aptamer.

Zhou et al. (2018), Theranostics, describes a similar construct with 2 uracils as the linker.

U.S. Pat. No. 8,916,696 discloses a “sticky bridge” construct where an aptamer is non-covalently conjugated to an siRNA. One pair of complementary GC rich sticky bridge sequences is attached to the 3′ end of the aptamer. The complement of this sequence is attached to one strand of the siRNA and joined by Watson-Crick base pairing.

U.S patent application U.S. Ser. No. 17/171,050 discloses a HER2 aptamer-EGFR siRNA-HER3 aptamer wherein the aptamer and siRNAs are separated by a four Adenine linker.

U.S. Pat. No. 10,689,654 discloses a PSMA aptamer-Survivin siRNA-EGFR siRNA-PSMA aptamer construct wherein the two siRNAs are separated by a four uracil linker.

U.S patent application U.S. Ser. No. 13/376,873 discloses a bispecific PSMA-4-1BB aptamer conjugate wherein a PSMA aptamer and a bivalent 4-1BB aptamer were tethered to complementary linker sequences and hybridized through Watson-Crick pairing.

Chu et al., Nucleic Acids Res, describes an anti-PSMA aptamer coupled to siRNA via a modular streptavidin bridge.

Huang et al., Chembiochem, describes an aptamer-doxorubicin conjugate formed via an acid labile linker. These linkers can cleave the release of the drug being delivered in the acidic environment of the endosome, a strategy that can be employed with siRNA as well.

U.S patent application U.S. Ser. No. 11/989,590 discloses a “kissing loop” structure containing a dimer of a chimeric phage (p)RNA-CD4 aptamer and chimeric pRNA-siRNA. The anti-CD4 aptamer or siRNAs were non-covalently joined via phi29 RNAs containing complementary loop domains. Through interactions of the interlocking left and right loops, chimeric phi29 RNAs are formed.

Antibody-siRNA chimeras have been used to deliver siRNAs to downregulate expression of gene targets (Lieberman et al., 2005)

U.S. Pat. No. 8,168,601 discloses an antibody-protamine fusion protein that binds siRNA when mixed in order to deliver the siRNA into cells expressing an antigen recognized by the antibody.

Xia et al., Mol. Pharmaceutics 2009, describes a receptor-specific monoclonal antibody bound to an siRNA via avidin-biotin binding.

Nanna et al., Nucleic Acids Res 2020, describes a dual variable domain (DVD) antibody with an inner Fv that contains a reactive lysine (Lys) residue that is unprotonated and highly nucleophilic at physiological pH and reacts specifically with β-lactam functionalized hapten derivatives. The antibody is reacted with symmetrical beta-lactam functionalized siRNAs.

Sugo et al., Controlled Release 2016, describes direct conjugation between CD71 Mab-siRNA. F(ab′)2 fragments were reduced with cysteamine to generate fragments with two thiol groups. Multiple structures of Thiol-reactive siRNAs were described and mixed with the purified F(ab).

In addition to antibody and aptamer delivery ligands, Folate, CpG, Centyrins, and Peptides are amongst other delivery ligands in development for RNAi conjugation (Abdelaal and Kasinski 2021).

PCT application PCT/US2007/026432 describes conjugating unmethylated CpG-motif (CpG ODN) and siRNAs. The ODN and antisense strands of siRNAs were linked using 6 units of C3 carbon chain linker. The resulting constructs were hybridized with complementary sense strands of siRNAs to create chimeric ODN-siRNA.

Orellana, Sci Transl Med 2017, describes Folate conjugated miR-34a achieved using DBCO-click chemistry.

Klein et al., Molecular Therapy 2021, describes centyrins conjugated to siRNA through cysteine-specific chemistry via maleimide handles on the siRNA as well as using the sortase reaction.

Lundberg et al, FASEB 2007, describes endosomolytic cell-penetrating peptides conjugated to siRNA covalently using a disulfide bridge; and noncovalently, where the CPP is co-incubated with the siRNA in a molar excess.

In spite of recent advances, there is a need in the art for compositions and methods of delivering anti-tumor agents to cells with enhanced efficacy and decreased toxicity. The nucleic acid compounds and methods of using the same as provided herein solve these and other problems in the art.

### SUMMARY OF THE INVENTION

In one aspect, the present invention provides a therapeutic composition comprising a ligand and a cytotoxin, wherein the cytotoxin is a siRNA construct.

### BRIEF DESCRIPTION OF THE DRAWINGS

FIGS. 1A-1G: depict the sequence alignment of UBBs1 to various targets, non-binding regions are highlighted.

FIG. 1A: depicts BLAST results of UBBs1 showing homologous regions to UBB mRNA at three regions with 19/19, 18/19 and 17/19 identity over the 19 nt stretch. Plus/Plus indicated that the guide strand of UBBs1 would bind the mRNA of UBB. FIG. 1A discloses SEQ ID NOS 12, 72, 12, and 73-77, respectively, in order of appearance.

FIG. 1B: depicts BLAST results of UBBs1 showing homologous regions to UBC mRNA at three regions with 14/14 identity over the 19 nt stretch. Results for UBBs1 BLAST showing binding to UBC mRNA with 14/14 identity. Further examination showed 3 of 4 nt were identical and overall 17/19 identity to UBBs1. FIG. 1B discloses SEQ ID NOS 78, 79, 78, 79, 78, 79, 76, and 77, respectively, in order of appearance.

FIG. 1C: depicts BLAST results of UBBs1 showing homologous regions to DCP2 mRNA at one region with 15/15 identity. FIG. 1C discloses SEQ ID NOS 80, 81, 76, and 77, respectively, in order of appearance.

FIG. 1D: depicts BLAST results of UBBs1 showing homologous regions to FAM83F mRNA at one region with 15/15 identity. FIG. 1D discloses SEQ ID NOS 82, 83, 76, and 77, respectively, in order of appearance.

FIG. 1E: depicts BLAST results of UBBs1 showing homologous regions to LOC646588 Mrna at one region with 15/15 identity. FIG. 1E discloses SEQ ID NOS 82, 83, 76, and 77, respectively, in order of appearance.

FIG. 1F: depicts BLAST results of UBBs1 showing homologous regions to NACA2 mRNA at one region with 15/15 identity. FIG. 1F discloses SEQ ID NOS 84, 85, 76, and 77, respectively, in order of appearance.

FIG. 1G: depicts BLAST results of UBBs1 showing homologous regions to RNF17 mRNA at one region with 15/15 identity. FIG. 1G discloses SEQ ID NOS 80, 81, 76, and 77, respectively, in order of appearance.

FIG. 2A: depicts the UBBs1 siRNA targeting sites (highlighted in yellow) on the UBB sequence. Nucleotide differences are highlighted in red (red indicated by solid lines) and similar repeat sequences are in blue (blue indicated by dotted lines). FIG. 2A discloses SEQ ID NO: 86.

FIG. 2B: depicts the UBBs1 siRNA targeting sites (highlighted in yellow) on the UBC sequence. Nucleotide differences are highlighted in red (red indicated by solid lines) and similar repeat sequences are in blue (blue indicated by dotted lines). FIG. 2A discloses SEQ ID NO: 86.

FIG. 3A depicts a template structure of an Aptamer-Sirna Chimera. FIG. 3A discloses SEQ ID NOS 88, 76, 77, and 88, respectively, in order of appearance.

FIG. 3B depicts a schematic of a dual UBB/UBC Sirna with Aptamers depicting Ubbs1 Sirna and EPCAM Aptamers. FIG. 3B discloses SEQ ID NOS 89 and 90, respectively, in order of appearance.

FIGS. 4A-B: HCT-116, SW480, RKO, and HT-29 Colon Cancer Cells were treated under standard Sirna transfection conditions with various Sirna compounds including those previously listed as well as ASN (Negative Control) and ASP (Positive Control) (16.7 nM; 96 Hr).

FIG. 4A depicts the results of HCT-116 cells treated with specified Sirnas under these conditions.

FIG. 4B depicts the results of SW480 cells treated with specified Sirnas under these conditions.

FIGS. 5A-B: HCT-116, SW480, RKO, and HT-29 Colon Cancer Cells were treated under standard Sirna transfection conditions with various Sirna compounds including those previously listed as well as ASN (Negative Control) and ASP (Positive Control) (16.7 nM; 96 Hr).

FIG. 5A depicts the results of HT-29 Cells treated with specified Sirnas under these conditions.

FIG. 5B depicts the results of RKO cells treated with specified Sirnas under these conditions.

FIGS. 6A-B: MCF-7 and SK-BR-3 breast cancer cells were treated under standard Sirna transfection conditions with various Sirna compounds including those previously listed as well as controls: ASN Sirna (Negative), ASP Sirna (Positive) (16.7 nM; 96 Hr).

FIG. 6A depicts the results of MCF-7 cells treated with specified Sirnas under these conditions.

FIG. 6B depicts the results of SK-BR-3 cells treated with specified Sirnas under these conditions.

FIGS. 7A-7B depicts the dose response of various Sirna sequences on colon cancer cells.

FIG. 7A: Dose Response Curve Of HCT-116 and various Sirna sequences. Cells were grown to 2,000 cells/well in a 384-well plate, and treated with 62 Pm-15 nM of compounds for 96 hours.

FIG. 7B: Dose Response Curve of SW480 and various Sirna sequences. Cells were grown to 2,000 cells/well in a 384-well plate, and treated with 62 Pm-15 nM of compounds for 96 hours.

FIGS. 8A-D: HT29, RKO, SW480, and HCT116 cells were treated with Sirna or Control (15 nM Sirna; 20 Hr).

FIG. 8A: HT29, RKO, SW480, and HCT116 cells treated with various Sirnas and UBB expression was measured, normalized by GAPDH.

FIG. 8B: HT29, RKO, SW480, and HCT116 cells treated with various Sirnas and UBC expression was measured, normalized by B-Actin.

FIG. 8C: HT29, RKO, SW480, and HCT116 cells treated with various Sirnas and UBB expression was measured, normalized by B-Actin.

FIG. 8D: HT29, RKO, SW480, and HCT116 cells treated with various Sirnas and UBC expression was measured, normalized by B-Actin.

FIG. 9: HCT116 cells were treated with the specified Sirna including U01, A Luciferase GL3 Sirna (15 nM Sirna; 20 Hr). Qpcr results were normalized to GAPDH.

FIG. 10: HCT116 cells were treated with the specified Sirna including U01, A Luciferase GL3 Sirna (15 nM Sirna; 20 Hr). Qpcr results were normalized to GAPDH.

FIGS. 11A-C: depicts BLAST results of homologous regions between UBB and UBC mRNA at regions with 19/19, 18/19 and 17/19 identity over the 19 Nt stretch.

FIG. 11A discloses SEQ ID NOS 91, 92, 43, 43, 54-58 and 46, respectively, in order of appearance.

FIG. 11B discloses SEQ ID NOS 93, 94, 44, 44, 45, 45, 56, 57, 59 and 48, respectively, in order of appearance.

FIG. 11C discloses SEQ ID NOS 95, 96, 44, 44, 11, 53, 12, 51, 46, 46, 47 and 47, respectively, in order of appearance.

FIGS. 12A-B: Treatment of cells with various dual UBB-UBC inhibitors.

FIG. 12A: HCT-116, a colon cancer cell line, were treated under standard Sirna transfection conditions with Sirna compounds targeting mRNA sequences previously listed as well as ASN (Negative Control) and ASP (Positive Control) (16.7 nM; 96 Hr). U32, U50, U51 are Negative Control Sirna's.

FIG. 12B: SK-BR3, a breast cancer cell line, was treated in the same manner.

FIGS. 13A-B: Human UBB and UBC sequences were compared to mouse in order to find homologous regions for in vivo work. FIGS. 13A-B disclose SEQ ID NOS 97, 98, 32, 101, 99, 100, 51, 102, and 103, respectively, in order of appearance.

FIG. 14: Gene expression of UBB and UBC in HCT116 cells was measured by Qpcr following Sirna treatment.

FIGS. 15A-D: 2′F Pyrimidine modifications of the Sirna targeting SEQ ID NO: 34.

FIGS. 15A: depicts the modifications on the passenger strand, U21Fp and discloses SEQ ID NOS 104 and 105, respectively, in order of appearance.

FIG. 15B: depicts the modifications on the guide strand, U21Fg and discloses SEQ ID NOS 104 and 105, respectively, in order of appearance.

FIG. 15C: depicts the unmodified U22ds siRNA and discloses SEQ ID NOS 106 and 107, respectively, in order of appearance.

FIG. 15D: depicts the U22ds siRNA with modifications on the guide strand and discloses SEQ ID NOS 61 and 107, respectively, in order of appearance.

FIG. 16: HCT-116 Cells were treated with modified and unmodified UBB-UBC targeting Sirnas.

FIG. 17: Cell viability of HCT-116 cells treated with modified and unmodified UBB-UBC targeting Sirnas was measured.

FIG. 18A: The structure of an Epcam Aptamer-UBB/UBC targeting Sirna-Epcam Aptamer Chimera is depicted. FIG. 18A discloses SEQ ID NOS 108 and 109, respectively, in order of appearance.

FIG. 18B: The Structure was synthesized, and the resulting products were run on a gel for confirmation.

### DETAILED DESCRIPTION OF THE INVENTION

In certain embodiments, a targeted RNA cytotoxin is provided that can be used in the place of the previously known cytotoxins used in ADCs, as well as in other novel therapeutics such as aptamer-siRNA chimeras. The targeted RNA cytotoxin can also be linked to other targeting agents such as aptamers, monoclonal antibodies, antibody fragments, cytokines, growth factors, peptides, or centryns. Centyrins are a new type of ligand useful in certain embodiments of the present invention. In further embodiments of the instant invention, a targeted cytotoxin platform is provided comprising a delivery agent and a cytotoxin that is processed to inhibit two or more oncogenes.

In certain embodiments, the cytotoxin is an siRNA that is processed by cellular RNAi machinery to produce two siRNAs that specifically inhibit expression of two or more different genes.

As used in this application and unless otherwise required by the context, the term “cytotoxin” is defined as a UBB/UBC inhibitor, wherein a singular molecule is able to functionally inhibit both UBB and UBC to create a cytotoxic effect in cancer cells.

In certain embodiments the cytotoxin is a single siRNA which is able to selectively inhibit UBB and UBC.

In certain embodiments siRNAs have been experimentally verified by real-time RT-PCR analysis and shown to provide at least 70% target knockdown at the mRNA level when used under optimal delivery conditions (confirmed using validated positive control and measured at the mRNA level 24 to 48 hours after transfection using 100 nM siRNA).

In certain other embodiments, siRNAs have been demonstrated to silence target gene expression by at least 75% at the mRNA level when used under optimal delivery conditions as validated by positive controls and measured at the mRNA level 24 to 48 hours after transfection using 100 nM siRNA.

Certain embodiments provide a bivalent siRNA chimera that is processed by cellular RNAi machinery to produce two siRNAs that specifically inhibit expression of UBB and UBC. Ubiquitin B (UBB) is one of the two genes that encode for Ubiquitin. Silencing of UBB results in dependence on the second gene, Ubiquitin C (UBC) (Tsherniak et al., Cell, 170: 564-576(2017)). Targeting of UBC in high-grade serous ovarian cancer (HGSOC), a cancer known for chronic UBB repression, demonstrated tumor regression and long term survival benefits. Thus, dual targeting UBB and UBC is useful as a therapeutic strategy for cancer (Kedves, et al., Clin Invest, 127: 4554-4568 (2017)).

In certain embodiments, the cytotoxin is conjugated to a delivery and targeting agent which binds to a cell surface protein expressed on cancer cells.

In certain embodiments, the cytotoxin is conjugated to a delivery agent which binds to epithelial cell adhesion molecules (EpCAM), a glycosylated membrane protein.

In certain embodiments, the cytotoxin is conjugated to a delivery agent which is an aptamer, monoclonal antibody, antibody fragment, cytokine, growth factor, peptides, or centryn.

In certain embodiments, the cytotoxin is conjugated to a delivery agent which binds to the gene product of ERBB2(HER2) (NCBI Gene ID: 2064). HER2, a membrane tyrosine kinase, is overexpressed in 20%-30% of breast cancer and correlates with poor prognosis, high aggressiveness, and extensive drug resistance. U.S. Pat. No. 10,960,086 discloses an aptamer targeting HER2 as part of an siRNA-aptamer chimera.

In certain embodiments, the cytotoxin is conjugated to a delivery agent which binds to the gene product of ERBB3(HER3) (NCBI Gene ID: 2065). HER3, a membrane tyrosine kinase, is involved in the resistance against EGFR- and HER2-targeted therapies through activation of a compensatory survival pathway. U.S. Pat. No. 10,960,086 discloses an aptamer targeting HER3 as part of an siRNA-aptamer chimera.

In certain embodiments, the cytotoxin is conjugated to a delivery agent which binds to the gene product of PSMA (NCBI Gene ID: 2346). Prostate-specific membrane antigen is a transmembrane protein expressed in all types of prostatic tissue.

In certain embodiments, the cytotoxin is conjugated to a delivery agent which binds to the gene product of CD44 (NCBI Gene ID: 960). CD44 is a transmembrane glycoprotein whose aberrant expression and dysregulation contributes to tumor initiation and progression. CD44 is involved in many processes including T cell differentiation, branching morphogenesis, proliferation, adhesion and migration. CD44 is a common biomarker of cancer stem cells.

In certain embodiments, the cytotoxin is conjugated to a delivery agent which binds to the gene product of EPCAM (NCBI Gene ID: 4072). EPCAM is a glycosylated membrane protein that is expressed in most organs and glands, with the highest expression in colon and is associated with colon cancer cell migration, proliferation, metastasis, and poor prognosis. A single EpCAM aptamer consisting of 19-nt RNA possesses similar binding affinity as antibodies and is efficiently internalized through receptor-mediated endocytosis (Shigdar, et al., Cancer Sci, 102:991-998 (2011).

In certain embodiments, the cytotoxin is conjugated to a delivery agent which binds to the gene product of PSCA, prostate stem cell antigen (NCBI Gene ID: 8000). PSCA is a membrane glycoprotein predominantly expressed in the prostate with a possible role in cell adhesion, proliferation control and cell survival. PSCA can have a tumor promoting or a tumor suppressive effect depending on the cell type.

In certain embodiments, the cytotoxin is conjugated to a delivery agent which binds to the gene product of PD1 (NCBI Gene ID: 5133). PD1 is an immune checkpoint molecule exploited by tumors to dampen T cell activation and avoid autoimmunity and the effects of an inflammatory response. Inhibiting PD1 enhances anti-tumor immunity.

In certain embodiments, the cytotoxin is conjugated to a delivery agent which binds to the gene product of CTLA4 (NCBI Gene ID: 1493). CTLA4 is an immune checkpoint molecule whose expression is dysregulated in tumors and in tumor-associated T cells. (Santulli-Marotto, S. et al., Cancer Res 63:7483-7489 (2003)). U.S. patent application Ser. No. 16/892,995 provides a CTLA-4 aptamer conjugated to an antisense nucleic acid.

In certain embodiments, the cytotoxin is conjugated to a delivery agent which binds to the gene product of TROP2 (NCBI Gene ID: 4070). TROP2, a cell-surface glycoprotein, is a paralog of epithelial-specific cell adhesion molecule (EpCAM). It is overexpressed in adenocarcinomas, minimally expressed in normal tissues, and expression level is correlated with tumor invasiveness and poor prognosis.

In certain embodiments, the cytotoxin is conjugated to a delivery agent which binds to the gene product of CD73 (NCBI Gene ID: 4907). CD73 is part of an enzyme cascade to breakdown ATP into adenosine. Overexpression of CD73 occurs in many cancers and leads to overproduction of adenosine which suppresses the antitumor immune response and helps aid cancer proliferation, angiogenesis, and metastasis.

In certain embodiments, the cytotoxin is conjugated to a delivery agent which binds to the gene product of LAG-3 (NCBI Gene ID: 3902). LAG3, cell surface molecule, is primarily expressed on activated T cells and NK cells and is a marker for the activation of CD4+ and CD8+ T cells. The co-expression of LAG3 with other inhibitory molecules including PD-1 induces T cell exhaustion.

In certain embodiments, the cytotoxin is conjugated to a delivery agent which binds to the gene product of TIM-3 (NCBI Gene ID: 84868). TIM-3, cell surface molecule, is constitutively expressed on innate immune cells and suppresses innate antitumor immunity by mediating T-cell exhaustion. TIM-3 is co-expressed with PD-1 and is upregulated during PD-1 blocking therapy. Blocking the TIM-3 pathway enhances cancer immunity and increases interferon-gamma (IFN-γ) in T cells.

In certain embodiments, a method is provided which includes administering to a subject in need thereof and effective amount of bivalent siRNA chimera having aptamers that specifically bind to EPCAM and siRNA constructs that are processed to produce siRNA that inhibits expression of UBB and UBC.

Unless stated otherwise, the following terms and phrases as used herein are intended to have the following meanings:

The term “antibody” herein is used in the broadest sense and specifically covers intact monoclonal antibodies, polyclonal antibodies, multispecific antibodies (e.g., bispecific antibodies) formed from at least two intact antibodies, and antibody fragments, so long as they exhibit the desired biological activity. The antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA, 81:6851-6855).

A “ligand” is defined herein as any molecule or atom that binds to or forms a complex with a receiving molecule, often proteins. Non-limiting examples of ligands include a monoclonal antibody, antibody mimics, antibody fragment, a centyrin, a cytokine, a growth factor, nucleic acid, nanoparticle, polymer, protein, aptamer, lipid, small molecule, radionucleotide, peptide, or a peptide fragment, although additional ligand technologies continue to be developed. Ligands according to the invention may also be called “delivery agents”.

Ligands include a Dextran cage, nanotube, quantum dot, magnetic nanoparticles, HPMA-s-APMA, PNIPAAm, PEG, Penatratin, Transportan, Tat, Anandamine, DAC, EPA, PC-DCA, a-tocopherol, cholesterol, GalNac, Lac, M6P, DUPA, Folate, CpG1668, LGRH peptide, cRGD, Tat-AHNP, Octreotide, IGF1 mimetic peptide, IL2, anti-CD22 dsFv, anti-CD25 scFv, GMCSF, Anti-CD-25 mAb, Anti-CD3 biFv, Anti-CD22 Fab, Anti-CD30 mAb, Anti-CD33 mAb, Variant IL3, Mesolthelin, cholesterol, EpCAM, EGFRvIII, EGFR, ErbB2, IL13R, IL4R, or TfR.

A ligand “which binds” an antigen of interest is one capable of binding that antigen with sufficient affinity such that the ligand is useful in targeting a cell expressing the antigen.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic.

The term “cytotoxic agent” as used herein refers to a substance that inhibits or prevents the function of cells and/or causes destruction of cells. The term “cytostatic agent” as used herein refers to a substance that inhibits or prevents the growth, proliferation and/or division of cells.

A nucleic acid is “operably linked” when it is placed into a functional relationship with another nucleic acid sequence.

The terms “treat” or “treatment,” unless otherwise indicated by context, refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In the context of cancer, the term “treating” includes any or all of preventing growth of tumor cells, cancer cells, or of a tumor; preventing replication of tumor cells or cancer cells, lessening of overall tumor burden or decreasing the number of cancerous cells, and ameliorating one or more symptoms associated with the disease.

In the context of an autoimmune disease, the term “treating” includes any or all of preventing replication of cells associated with an autoimmune disease state including, but not limited to, cells that produce an autoimmune antibody, lessening the autoimmune-antibody burden and ameliorating one or more symptoms of an autoimmune disease.

In the context of an infectious disease, the term “treating” includes any or all of preventing the growth, multiplication or replication of the pathogen that causes the infectious disease and ameliorating one or more symptoms of an infectious disease.

Methods according to the invention include administering a dual targeting siRNA agent to the subject to be treated. When the organism to be treated is a mammal such as a human, the composition can be administered by any means known in the art including, but not limited to oral or parenteral routes, including intravenous, intramuscular, subcutaneous, transdermal, and airway (aerosol) administration. In some embodiments, the compositions are administered by intravenous infusion or injection.

An antibody according to the invention can also be a bispecific antibody. For further details for generating bispecific antibodies see, for example, Suresh et al., Methods in Enzymology, 1986, 121:210; Rodrigues et al., 1993, J. of Immunology 151:6954-6961; Carter et al., 1992, Bio/Technology 10:163-167; Carter et al., 1995, J. of Hematotherapy 4:463-470; Merchant et al., 1998, Nature Biotechnology 16:677-681, the disclosure of which is hereby specifically incorporated herein.

In other embodiments, the antibody is a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, preferably at least 10, 20 or 50 amino acid portion of the protein) that is not the antibody. Preferably, the antibody or fragment thereof is covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

In certain embodiments, known antibodies for the treatment or prevention of cancer can be used. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody, HERCEPTIN® (trastuzumab; Genentech) for the treatment of patients with metastatic breast cancer; RITUXAN® (rituximab; Genentech) which is a chimeric anti-CD20 monoclonal antibody for the treatment of patients with non-Hodgkin's lymphoma; OvaRex (AltaRex Corporation, MA), which is a murine antibody for the treatment of ovarian cancer; Panorex (Glaxo Wellcome, NC) which is a murine IgG2a antibody for the treatment of colorectal cancer; Cetuximab Erbitux (Imclone Systems Inc., NY), which is an anti-EGFR IgG chimeric antibody for the treatment of epidermal growth factor positive cancers, such as head and neck cancer; Vitaxin (MedImmune, Inc., MD), which is a humanized antibody for the treatment of sarcoma; Campath I/H (Leukosite, MA), which is a humanized IgG1 antibody for the treatment of chronic lymphocytic leukemia (CLL); Smart MI95 (Protein Design Labs, Inc., CA), which is a humanized anti-CD33 IgG antibody for the treatment of acute myeloid leukemia (AML); LymphoCide (Immunomedics, Inc., NJ), which is a humanized anti-CD22 lgG antibody for the treatment of non-Hodgkin's lymphoma; Smart ID10 (Protein Design Labs, Inc., CA), which is a humanized anti-HLA-DR antibody for the treatment of non-Hodgkin's lymphoma; Oncolym (Techniclone, Inc., CA) which is a radiolabeled murine anti-HLA-Dr10 antibody for the treatment of non-Hodgkin's lymphoma; Allomune (BioTransplant, CA), which is a humanized anti-CD2 mAb for the treatment of Hodgkin's Disease or non-Hodgkin's lymphoma; Avastin (Genentech, Inc., CA), which is an anti-VEGF humanized antibody for the treatment of lung and colorectal cancers; Epratuzamab (Immunomedics, Inc., NJ and Amgen, Calif.) which is an anti-CD22 antibody for the treatment of non-Hodgkin's lymphoma; and CEAcide (Immunomedics, NJ), which is a humanized anti-CEA antibody for the treatment of colorectal cancer. Other ligands useful in the treatment of cancer include, but are not limited to, ligands against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1-KLH (breast cancer), CEA (colorectal), gp100 (melanoma), MART1 (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies according to the invention include, but are not limited to, BR96 mAb (Trail, P. A., Willner, D., Lasch, S. J., Henderson, A. J., Hofstead, S. J., Casazza, A. M., Firestone, R. A., Hellstrom, I., Hellstrom, K. E., “Cure of Xenografted Human Carcinomas by BR96-Doxorubicin Immunoconjugates” Science 1993, 261, 212-215), BR64 (Trail, P A, Willner, D, Knipe, J., Henderson, A. J., Lasch, S. J., Zoeckler, M. E., Trailsmith, M. D., Doyle, T. W., King, H. D., Casazza, A. M., Braslawsky, G. R., Brown, J. P., Hofstead, S. J., (Greenfield, R. S., Firestone, R. A., Mosure, K., Kadow, D. F., Yang, M. B., Hellstrom, K. E., and Hellstrom, I. “Effect of Linker Variation on the Stability, Potency, and Efficacy of Carcinoma-reactive BR64-Doxorubicin Immunoconjugates” Cancer Research 1997, 57, 100-105, mAbs against the CD40 antigen, such as S2C6 mAb (Francisco, J. A., Donaldson, K. L., Chace, D., Siegall, C. B., and Wahl, A. F. “Agonistic properties and in vivo antitumor activity of the anti-CD-40 antibody, SGN-14” Cancer Res. 2000, 60, 3225-3231), mAbs against the CD70 antigen, such as 1F6 mAb and 2F2 mAb, and mAbs against the CD30 antigen, such as AC10 (Bowen, M. A., Olsen, K. J., Cheng, L., Avila, D., and Podack, E. R. “Functional effects of CD30 on a large granular lymphoma cell line YT” J. Immunol., 151, 5896-5906, 1993: Wahl et al., 2002 Cancer Res. 62(13):3736-42). Many other internalizing antibodies that bind to tumor associated antigens can be used and have been reviewed (Franke, A. E., Sievers, E. L., and Scheinberg, D. A., “Cell surface receptor-targeted therapy of acute myeloid leukemia: a review” Cancer Biother Radiopharm. 2000, 15, 459-76; Murray, J. L., “Monoclonal antibody treatment of solid tumors: a coming of age” Semin Oncol. 2000, 27, 64-70; Breitling, F., and Dubel, S., Recombinant Antibodies, John Wiley, and Sons, New York, 1998). The disclosure of all of the above articles is hereby specifically incorporated herein by reference.

In another embodiment, known antibodies for the treatment or prevention of an autoimmune disease may be used in accordance with the compositions and methods of the invention. Antibodies immunospecific for an antigen of a cell that is responsible for producing autoimmune antibodies can be obtained or produced by any method known to one of skill in the art such as, e.g., chemical synthesis or recombinant expression techniques. In another embodiment, useful antibodies are immunospecific for the treatment of autoimmune diseases include, but are not limited to, Anti-Nuclear antibody; Anti-ds DNA; Anti-ss DNA, Anti-Cardiolipin antibody IgM, IgG; Anti-Phospholipid antibody IgM, IgG; Anti-SM antibody; Anti-Mitochondrial antibody; Thyroid antibody; Microsomal antibody; Thyroglobulin antibody; Anti-SCL-70; Anti-Jo; Anti-U.sub. 1RNP; Anti-La/SSB; Anti SSA; Anti-SSB; Anti-Perital Cells antibody; Anti-Histones; Anti-RNP; C-ANCA; P-ANCA; Anti centromere; Anti-Fibrillarin, and Anti-GBM antibody.

In certain embodiments, useful ligands can bind to both a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD19, CD22, CD28, CD79, CD90, CD152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD11a, CD11b, CD11c, CD18, CD29, CD41, CD49a, CD49b, CD49c, CD49d, CD49e, CD49f, CD103, and CD104. Non-limiting examples of suitable lectins are C-type, S-type, and I-type lectin.

In one embodiment, the ligand binds to an activated lymphocyte that is associated with an autoimmune disease.

In another specific embodiment, useful ligands immunospecific for a viral or a microbial antigen are monoclonal antibodies. The antibodies may be chimeric, humanized or human monoclonal antibodies. As used herein, the term “viral antigen” includes, but is not limited to, any viral peptide, polypeptide protein (e.g., HIV gp120, HIV nef, RSV F glycoprotein, influenza virus neuraminidase, influenza virus hemagglutinin, HTLV tax, herpes simplex virus glycoprotein (e.g., gB, gC, gD, and gE) and hepatitis B surface antigen) that is capable of eliciting an immune response.

Ligands which may be useful in the treatment of cancer include ligands against tumor-associated antigens (TAA). Such tumor-associated antigens are known in the art, and can prepared for use in generating ligands using methods and information which are well known in the art.

The compounds of certain embodiments of the invention are useful for treating cancer, an autoimmune disease or an infectious disease in a patient or useful as an intermediate for the synthesis of a cytotoxin-linker, cytotoxin-linker-ligand conjugate, and cytotoxin-ligand conjugate.

In another aspect, compositions are provided including an effective amount of a cytotoxin-linker-ligand conjugate and a pharmaceutically acceptable carrier or vehicle.

In still another aspect, the invention provides pharmaceutical compositions comprising an effective amount of a cytotoxin-linker compound and a pharmaceutically acceptable carrier or vehicle.

In still another aspect, the invention provides compositions comprising an effective amount of a cytotoxin-ligand conjugate and a pharmaceutically acceptable carrier or vehicle.

In yet another aspect, the invention provides methods for killing or inhibiting the multiplication of a tumor cell or cancer cell including administering to a patient in need thereof an effective amount of a cytotoxin-linker compound.

In another aspect, the invention provides methods for killing or inhibiting the multiplication of a tumor cell or cancer cell including administering to a patient in need thereof an effective amount of a cytotoxin-linker-ligand conjugate.

In another aspect, the invention provides methods for killing or inhibiting the multiplication of a tumor cell or cancer cell including administering to a patient in need thereof an effective amount of a cytotoxin-ligand conjugate.

In still another aspect, the invention provides methods for treating cancer including administering to a patient in need thereof an effective amount of a cytotoxin-linker compound.

In yet another aspect, the invention provides methods for treating cancer including administering to a patient in need thereof an effective amount of a cytotoxin-linker-ligand conjugate.

In yet another aspect, the invention provides methods for treating cancer including administering to a patient in need thereof an effective amount of a cytotoxin.

In still another aspect, the invention provides methods for killing or inhibiting the replication of a cell that expresses an autoimmune antibody including administering to a patient in need thereof an effective amount of a cytotoxin-linker compound.

In another aspect, the invention provides methods for killing or inhibiting the replication of a cell that expresses an autoimmune antibody including administering to a patient in need thereof an effective amount of a cytotoxin-linker-ligand conjugate.

In another aspect, the invention provides methods for killing or inhibiting the replication of a cell that expresses an autoimmune antibody including administering to a patient in need thereof an effective amount of a cytotoxin-ligand conjugate.

In yet another aspect, the invention provides methods for treating an autoimmune disease including administering to a patient in need thereof an effective amount of a cytotoxin-linker compound.

In yet another aspect, the invention provides methods for treating an autoimmune disease including administering to a patient in need thereof an effective amount of a cytotoxin-linker-ligand conjugate.

In yet another aspect, the invention provides methods for treating an autoimmune disease including administering to a patient in need thereof an effective amount of a cytotoxin-ligand conjugate.

In still another aspect, the invention provides methods for treating an infectious disease including administering to a patient in need thereof an effective amount of a cytotoxin-linker compound.

In still another aspect, the invention provides methods for treating an infectious disease including administering to a patient in need thereof an effective amount of a cytotoxin-linker-ligand conjugate.

In still another aspect, the invention provides methods for treating an infectious disease including administering to a patient in need thereof an effective amount of a cytotoxin-ligand conjugate.

In yet another aspect, the invention provides methods for preventing the multiplication of a tumor cell or cancer cell including administering to a patient in need thereof an effective amount of a cytotoxin-linker compound.

In another aspect, the invention provides methods for preventing the multiplication of a tumor cell or cancer cell including administering to a patient in need thereof an effective amount of a cytotoxin-linker-ligand conjugate.

In another aspect, the invention provides methods for preventing the multiplication of a tumor cell or cancer cell including administering to a patient in need thereof an effective amount of a cytotoxin-ligand conjugate.

In still another aspect, the invention provides methods for preventing cancer including administering to a patient in need thereof an effective amount of a cytotoxin-linker compound.

In yet another aspect, the invention provides methods for preventing cancer including administering to a patient in need thereof an effective amount of a cytotoxin-linker-ligand conjugate.

In yet another aspect, the invention provides methods for preventing cancer including administering to a patient in need thereof an effective amount of a cytotoxin-ligand conjugate.

In another aspect, a cytotoxin-linker compound is provided which can be used as an intermediate for the synthesis of a cytotoxin-linker-ligand conjugate.

In certain embodiments the ligand referred to above is an antibody.

In certain embodiments the ligand referred to above is an aptamer.

In certain embodiments the cytotoxin referred to above is an siRNA.

In certain embodiments the cytotoxin referred to above is an siRNA that inhibits both UBC and UBC.

In one embodiment, ligand is a ligand which binds to one or more of the following receptors: BMPR1B; E16; STEAP1; 0772P; MPF; Napi3b; Sema 5b; PSCA hlg; Endothelin type B receptor; RNF124; STEAP2 TrpM4; CRIPTO; CD21; CD79b; FcRH2; HER2; NCA; MDP; IL20R.alpha; Brevican; Ephb2R; ASLG659; PSCA; GEDA; BAFF-R; CD22; CD79a; CXCR5; HLA-DOB; P2X5; CD72; LY64; FCRH1; or IRTA2.

In another aspect, a cytotoxin-linker compound is provided which can be used as an intermediate for the synthesis of a cytotoxin-linker-ligand conjugate.

In another aspect, the ligand of the ligand-cytotoxin conjugate of the invention specifically binds to a receptor encoded by an ErbB2 gene.

In another aspect, the ligand is an antibody and the antibody of the antibody-cytotoxin conjugate is a humanized antibody selected from huMAb4D5-1, huMAb4D5-2, huMAb4D5-3, huMAb4D5-4, huMAb4D5-5, huMAb4D5-6, huMAb4D5-7 and huMAb4D5-8 (Trastuzurnab).

In another aspect, the invention includes a method for the treatment of cancer in a mammal, wherein the cancer is characterized by the overexpression of an ErbB2 receptor and does not respond, or responds poorly, to treatment with an anti-ErbB2 antibody, comprising administering to the mammal a therapeutically effective amount of a ligand-cytotoxin conjugate compound of the invention.

In another aspect, a substantial amount of the cytotoxin moiety is not cleaved from the ligand until the ligand-cytotoxin conjugate compound enters a cell with a cell-surface receptor specific for the ligand of the ligand-cytotoxin conjugate, and the cytotoxin moiety is cleaved from the ligand when the ligand-cytotoxin conjugate does enter the cell.

In another aspect, the bioavailability of the ligand-cytotoxin conjugate compound or an intracellular metabolite of the compound in a mammal is improved when compared to a therapeutic compound comprising the cytotoxin moiety of the ligand-cytotoxin conjugate compound, or when compared to an analog of the compound not having the cytotoxin moiety.

In another aspect, the cytotoxin moiety is intracellularly cleaved in a mammal from the ligand of the compound, or an intracellular metabolite of the compound.

In another aspect, the invention includes a pharmaceutical composition comprising an effective amount of the ligand-cytotoxin conjugate compound of the invention, or a pharmaceutically acceptable salt thereof, and a pharmaceutically acceptable diluent, carrier or excipient. The composition may further comprise a therapeutically effective amount of chemotherapeutic agent such as a tubulin-forming inhibitor, a topoisomerase inhibitor, and a DNA binder.

In another aspect, the invention includes an article of manufacture comprising an aptamer-cytotoxin conjugate compound of the invention; a container; and a package insert or label indicating that the compound can be used to treat cancer characterized by the overexpression of an ErbB2 receptor.

In another aspect, the invention includes a method for the treatment of cancer in a mammal, wherein the cancer is characterized by the overexpression of an ErbB2 receptor and does not respond, or responds poorly, to treatment with an anti-ErbB2 aptamer, comprising administering to the mammal a therapeutically effective amount of an aptamer-cytotoxin conjugate compound of the invention.

In another aspect, a substantial amount of the cytotoxin moiety is not cleaved from the aptamer until the aptamer-cytotoxin conjugate compound enters a cell with a cell-surface receptor specific for the aptamer of the aptamer-cytotoxin conjugate, and the cytotoxin moiety is cleaved from the aptamer when the aptamer-cytotoxin conjugate does enter the cell.

In another aspect, the bioavailability of the aptamer-cytotoxin conjugate compound or an intracellular metabolite of the compound in a mammal is improved when compared to a therapeutic compound comprising the cytotoxin moiety of the aptamer-cytotoxin conjugate compound, or when compared to an analog of the compound not having the cytotoxin moiety.

In another aspect, the cytotoxin moiety is intracellularly cleaved in a mammal from the aptamer of the compound, or an intracellular metabolite of the compound.

In another aspect, the invention includes a pharmaceutical composition comprising an effective amount of the aptamer-cytotoxin conjugate compound of the invention, or a pharmaceutically acceptable salt thereof, and a pharmaceutically acceptable diluent, carrier or excipient.

In another aspect, the invention includes a method for killing or inhibiting the proliferation of tumor cells or cancer cells comprising treating tumor cells or cancer cells with an amount of the aptamer-cytotoxin conjugate compound of the invention, or a pharmaceutically acceptable salt or solvate thereof, being effective to kill or inhibit the proliferation of the tumor cells or cancer cells.

In another aspect, the invention includes a method of inhibiting cellular proliferation comprising exposing mammalian cells in a cell culture medium to an aptamer cytotoxin conjugate compound of the invention, wherein the aptamer cytotoxin conjugate compound enters the cells and the cytotoxin is cleaved from the remainder of the aptamer cytotoxin conjugate compound; whereby proliferation of the cells is inhibited.

In another aspect, the invention includes a method of treating cancer comprising administering to a patient a formulation of an aptamer-cytotoxin conjugate compound of the invention and a pharmaceutically acceptable diluent, carrier or excipient.

In another aspect, the invention includes an assay for detecting cancer cells comprising: (a) exposing cells to an aptamer-cytotoxin conjugate compound of the invention; and (b) determining the extent of binding of the aptamer-cytotoxin conjugate compound to the cells.

A further embodiment is an antibody cytotoxin conjugate (ADC), or an aptamer cytotoxin conjugate, or a pharmaceutically acceptable salt or solvate thereof, wherein Ab or aptamer (Ap) is an antibody or aptamer that binds a tumor associated antigen (a “TAA compound”).

Another embodiment is the TAA compound or pharmaceutically acceptable salt or solvate thereof that is in isolated and purified form.

Another embodiment is a method for killing or inhibiting the multiplication of a tumor cell or cancer cell comprising administering to a patient, for example a human with a hyperproliferative disorder, an amount of the TAA compound or a pharmaceutically acceptable salt or solvate thereof, said amount being effective to kill or inhibit the multiplication of a tumor cell or cancer cell.

Another embodiment is a method for treating cancer comprising administering to a patient, for example a human with a hyperproliferative disorder, an amount of the TAA compound or a pharmaceutically acceptable salt or solvate thereof, said amount being effective to treat cancer, alone or together with an effective amount of an additional anticancer agent.

Another embodiment is a method for treating an autoimmune disease, comprising administering to a patient, for example a human with a hyperproliferative disorder, an amount of the TAA compound or a pharmaceutically acceptable salt or solvate thereof, said amount being effective to treat an autoimmune disease.

The antibodies or aptamers suitable for use in the invention can be produced by any method known in the art for the synthesis of antibodies, in particular, by chemical synthesis or by recombinant expression, and are preferably produced by recombinant expression techniques.

Antibodies of the invention can be produced using any method known in the art to be useful for the synthesis of antibodies, in particular, by chemical synthesis or by recombinant expression.

Aptamers of the invention can be produced using any method known in the art to be useful for the synthesis of aptamers, in particular, by chemical synthesis or by recombinant expression.

In other embodiments, described is a composition including an effective amount of an Exemplary compound and/or Exemplary conjugate and a pharmaceutically acceptable carrier or vehicle. For convenience, the cytotoxin units and cytotoxin-linker compounds can be referred to as Exemplary compounds, while cytotoxin-ligand conjugates and cytotoxin-linker-ligand conjugates can be referred to as Exemplary conjugates. The compositions are suitable for veterinary or human administration.

### EXAMPLES

### Example 1: Identifying Target Gene with Multiple Target Regions

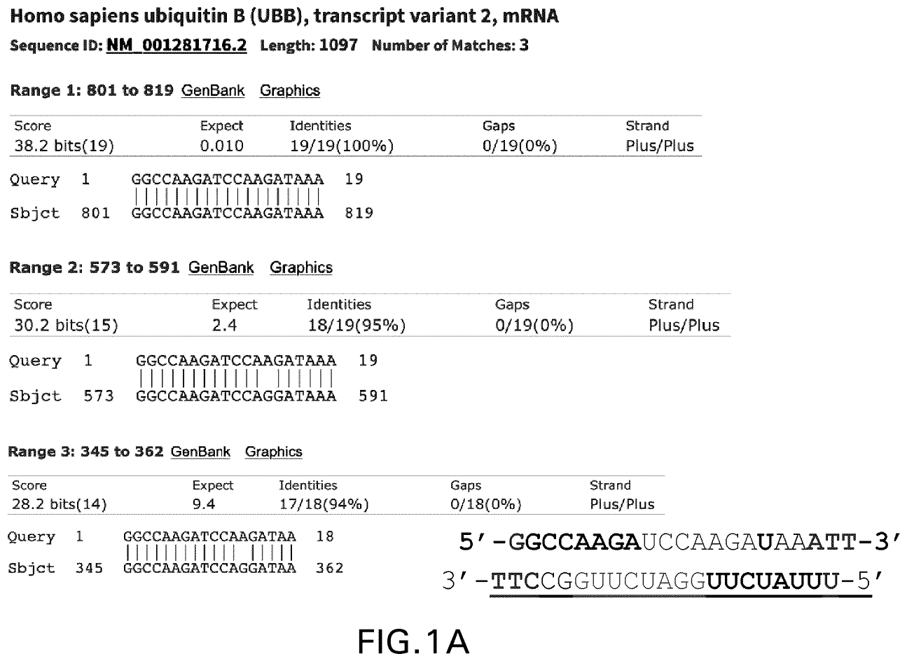

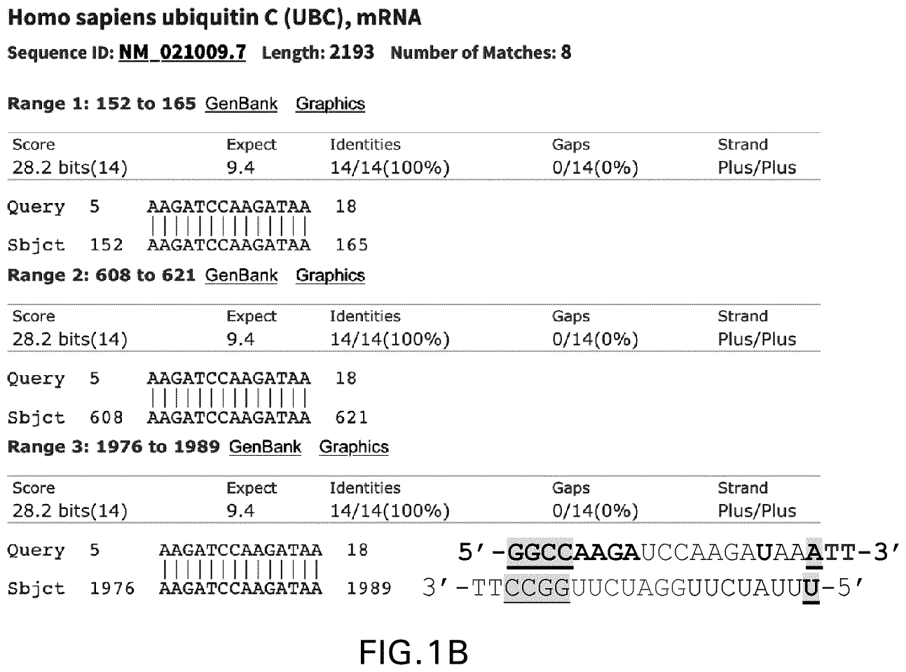

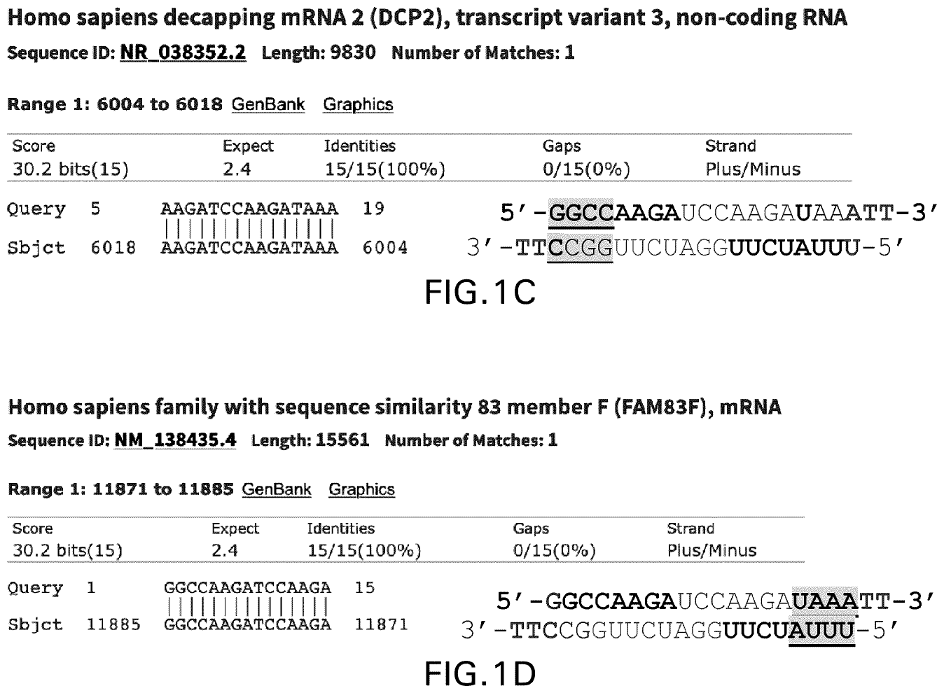

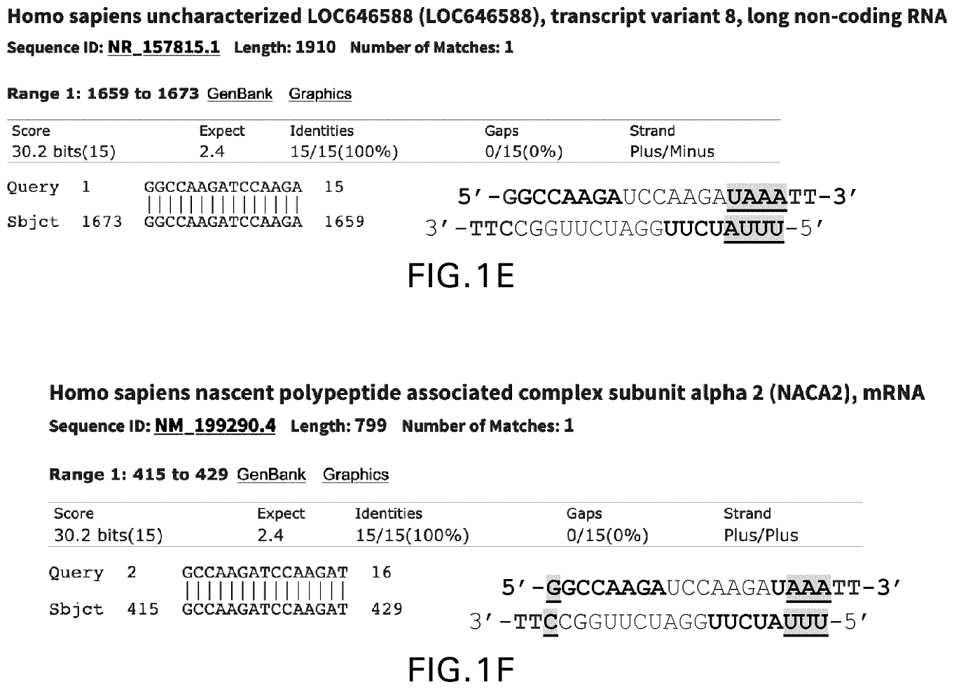

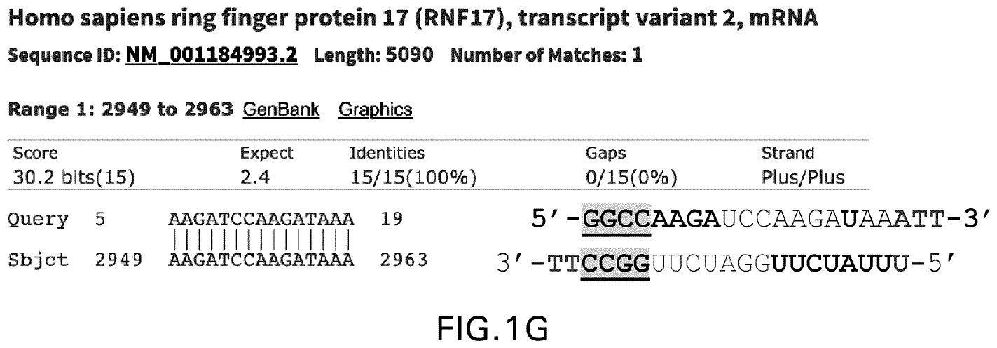

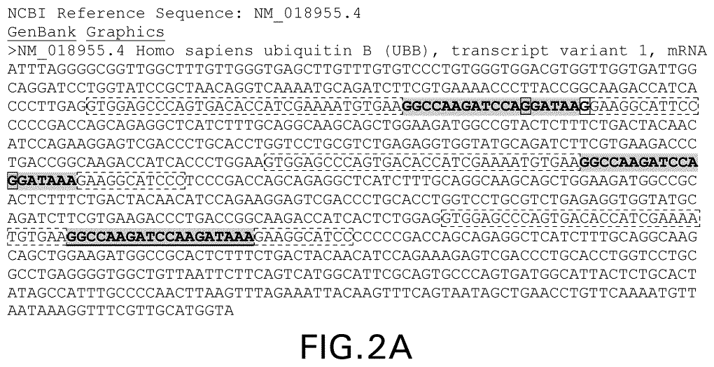

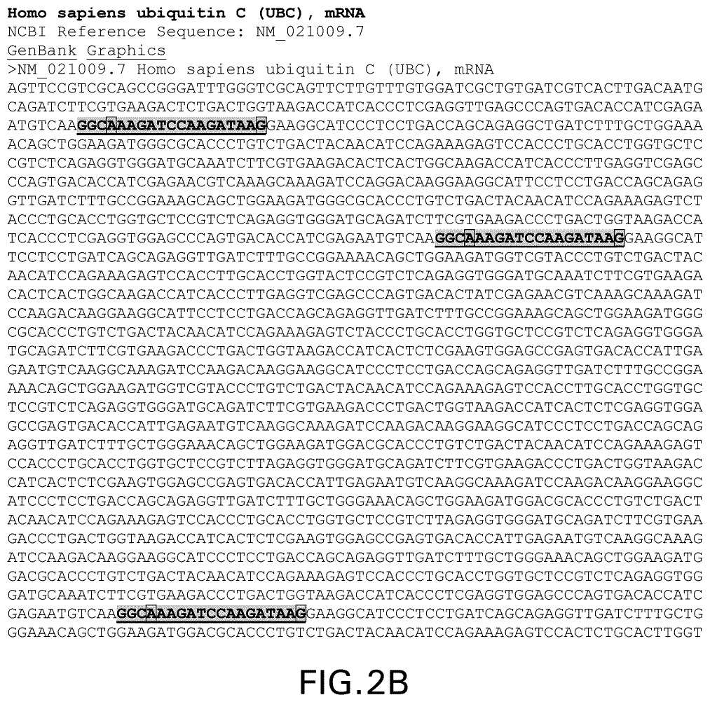

siRNA targeting sequences UBBs1- (SEQ ID NO: 1): AAGGCCAAGATCCAAGATAAA (U.S. Pat. No. 8,470,998) and UBBs2- (SEQ ID NO: 2): AAGAGGTGGTATGCAGATCTT. Utilizing Basic Local Alignment Search Tool (Blast) from the National Center for Biotechnology Information, UBB was found to have three targeting regions for UBBs1 with 19/19, 18/19, and 17/19 conserved identities (FIG. 1A and FIG. 2A). Thus, UBB is a useful gene for a siRNA to target in multiple regions.

FIG. 1: depicts the sequence alignment of UBBs1 to various targets, non-binding regions are highlighted.

FIG. 1A: depicts BLAST results of UBBs1 showing homologous regions to UBB mRNA at three regions with 19/19, 18/19 and 17/19 identity over the 19 nt stretch. Plus/Plus indicated that the guide strand of UBBs1 would bind the mRNA of UBB.

FIG. 1B: depicts BLAST results of UBBs1 showing homologous regions to UBC mRNA at three regions with 14/14 identity over the 19 nt stretch. Results for UBBs1 BLAST showing binding to UBC mRNA with 14/14 identity. Further examination showed 3 of 4 nt were identical and overall, 17/19 identity to UBBs1.

FIG. 1C: depicts BLAST results of UBBs1 showing homologous regions to DCP2 mRNA at one region with 15/15 identity.

FIG. 1D. depicts BLAST results of UBBs1 showing homologous regions to FAM83F mRNA at one region with 15/15 identity.

FIG. 1E. depicts BLAST results of UBBs1 showing homologous regions to LOC646588 mRNA at one region with 15/15 identity.

FIG. 1F. depicts BLAST results of UBBs1 showing homologous regions to NACA2 mRNA at one region with 15/15 identity.

FIG. 1G. depicts BLAST results of UBBs1 showing homologous regions to RNF17 mRNA at one region with 15/15 identity.

### Example 2: Identification of a UBBs1 Dual Target

After identifying a lead siRNA that bound to the UBB gene in three regions, 6 genes were identified through blast to have conserved homology with UBB to be a dual target partner to the siRNA inhibition. These targets (DCP2, FAM83F, LOC646588, RNF17, NACA2, and UBC, FIG. 1) were analyzed with the goal of finding key cancer dependencies. Analysis revealed that all but one were non-essential. UBC was found to be essential and a dual target of siRNA targeting UBBs1.

FIG. 2A: depicts the UBBs1 siRNA targeting sites (highlighted in yellow) on the UBB sequence. Nucleotide differences are highlighted in red and similar repeat sequences are in blue.

FIG. 2B: depicts the UBBs1 siRNA targeting sites (highlighted in yellow) on the UBC sequence. Nucleotide differences are highlighted in red and similar repeat sequences are in blue.

### Example 3: Characterization of UBB/UBC Dual Inhibition

BLAST results of UBC (FIG. 1b) reveal three targeting regions for siRNA targeting UBBs1 all with 18/19 identify with a 14/14 identity stretch (FIG. 1B and FIG. 2B). These results point to one siRNA potentially targeting multiple genes and multiple regions within each gene (multi-multi-targeting).

### Example 4: A Schematic of a Dual UBB/UBC siRNA with Aptamers Depicting UBBs1 siRNA and EPCAM Aptamers

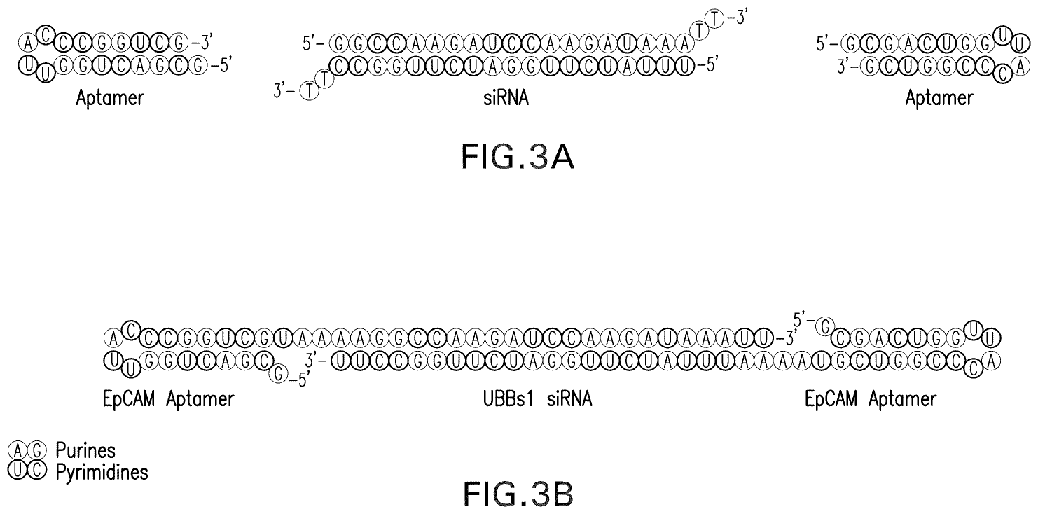

A lead siRNA or aptamer compound could be substituted in this template (FIG. 3A). A depiction of an aptamer-siRNA chimera with EPCAM aptamers and UBBs1 siRNA combined with an example of an acceptable linker, for example as disclosed in U.S. Pat. No. 10,960,086 (FIG. 3B).

### Example 5: Library Development of UBBs1 Variations

A siRNA library was developed containing 19 compounds of 19mer siRNA's targeting UBB Sequences:

(SEQ ID NO: 3):
5′-AAATGTGAAGGCCAAGATC-3′
(SEQ ID NO: 4):
5′-AATGTGAAGGCCAAGATCC-3′
(SEQ ID NO: 5):
5′-ATGTGAAGGCCAAGATCCA-3′
(SEQ ID NO: 6):
5′-TGTGAAGGCCAAGATCCAA-3′
(SEQ ID NO: 7):
5′-GTGAAGGCCAAGATCCAAG-3′
(SEQ ID NO: 8):
5′-TGAAGGCCAAGATCCAAGA-3′
(SEQ ID NO: 9):
5′-GAAGGCCAAGATCCAAGAT-3′
(SEQ ID NO: 10):
5′-AAGGCCAAGATCCAAGATA-3′
(SEQ ID NO: 11):
5′-AGGCCAAGATCCAAGATAA-3′
(SEQ ID NO: 12):
5′-GGCCAAGATCCAAGATAAA-3′
(SEQ ID NO: 13):
5′-GCCAAGATCCAAGATAAAG-3′
(SEQ ID NO: 14):
5′-CCAAGATCCAAGATAAAGA-3′
(SEQ ID NO: 15):
5′-CAAGATCCAAGATAAAGAA-3′
(SEQ ID NO: 16):
5′-AAGATCCAAGATAAAGAAG-3′
(SEQ ID NO: 17):
5′-AGATCCAAGATAAAGAAGG-3′
(SEQ ID NO: 18):
5′-GATCCAAGATAAAGAAGGC-3′
(SEQ ID NO: 19):
5′-ATCCAAGATAAAGAAGGCA-3′
(SEQ ID NO: 20):
5′-TCCAAGATAAAGAAGGCAT-3′
(SEQ ID NO: 21):
5′-CCAAGATAAAGAAGGCATC-3′
(SEQ ID NO: 22):
5′-CAGGATCCTGGTATCCGCTAA-3′ (UBB_1)
(SEQ ID NO: 23):
5′-ATGGCATTACTCTGCACTATA-3′ (UBB_2)
(SEQ ID NO: 24):
5′-CCAACTTAAGTTTAGAAATTA-3′ (UBB_3)
(SEQ ID NO: 25):
5′-GAGGCTCATCTTTGCAGGCAA-3′ (UBB_4)

Utilizing siRNA Wizard Software (InvivoGen), 6 scrambled UBBs1 targeting sequences were developed as controls:

(SEQ ID NO: 26):
5′-GAACAACCGGCAAATAGAT-3′
(SEQ ID NO: 27):
5′-GCAATACGCGAAGACATAA-3′
(SEQ ID NO:: 28):
5′-GAAAGACGGACCATAACAT-3′
(SEQ ID NO: 29):
5′-GAAGAACCACGAAGACTTA-3′
(SEQ ID NO: 30):
5′-GTAGGACGCACAAACTAAA-3′
(SEQ ID NO: 31):
5′-GGACAGATCGCTAAACAAA-3′

Three UBBs1-like targeting compounds were developed including one that is designed to target UBC in a conserved location to likely target both UBB and UBC.

UBBs1b (UBBs1-like) (SEQ ID NO: 32): 5′-GGCCAAGATCCAGGATAAA-3′ UBBs1c (UBBs1-like) (SEQ ID NO: 33): 5′-GGCCAAGATCCAGGATAAG-3′ UBC siRNA (UBBs1-like) (SEQ ID NO: 34): 5′-GGCAAAGATCCAAGATAAG-3′

### Example 6: In Vitro Validation of UBB siRNA Library

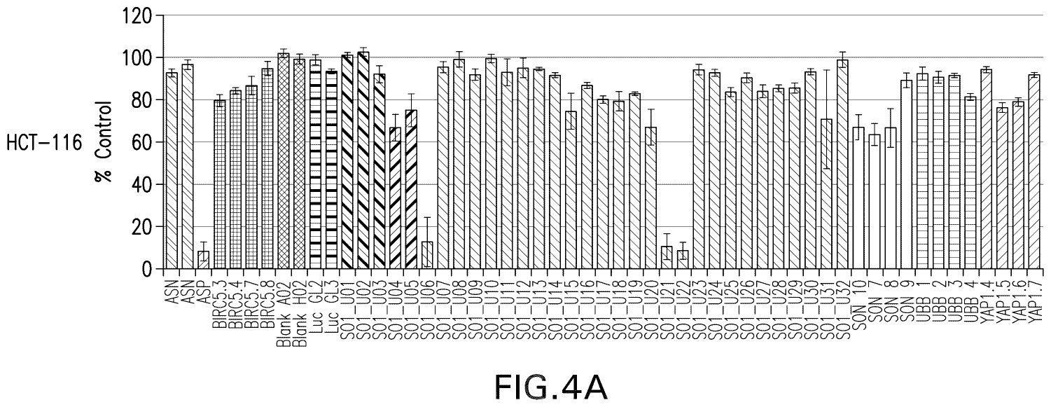

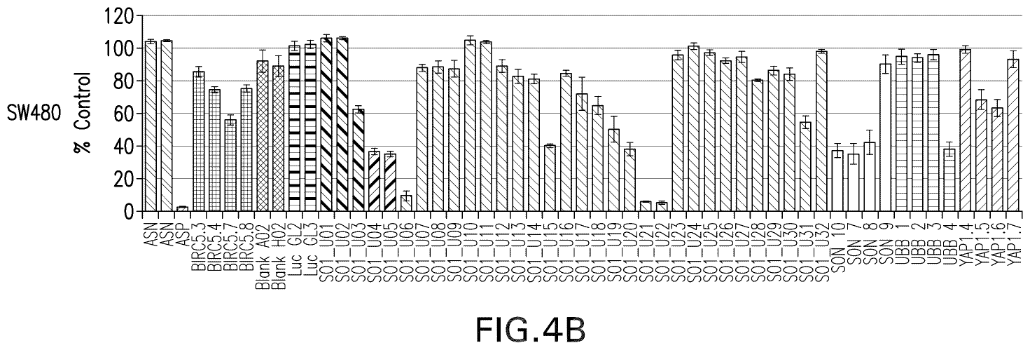

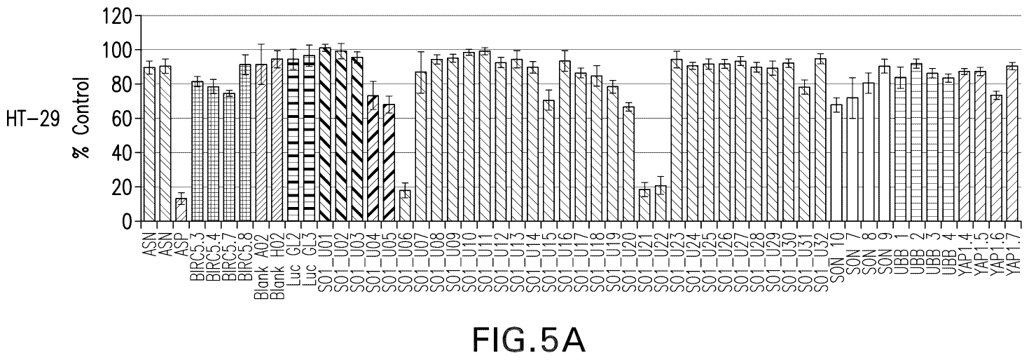

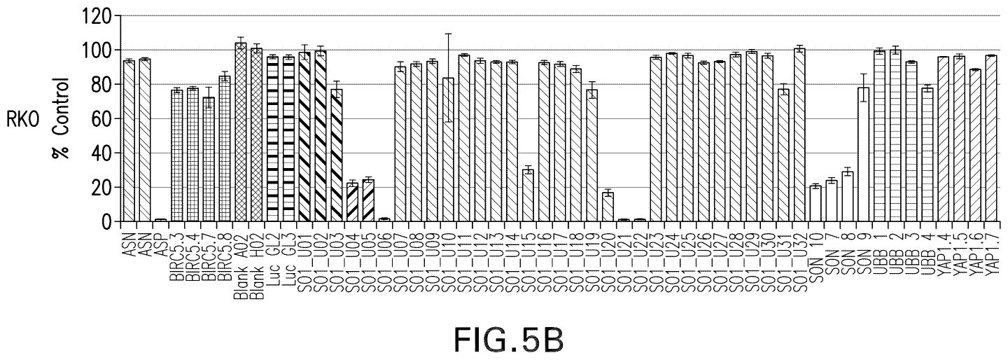

HCT-116, SW480, RKO, and HT-29 colon cancer cells were treated under standard siRNA transfection conditions with various siRNA compounds including those previously listed as well as ASN (negative control) and ASP (positive control) (16.7 nM; 96 hr) (FIGS. 4 and 5).

The siRNA targeting UBBs1 (SEQ ID NO: 12) is cytotoxic to SW480 and HCT-116. The siRNA targeting UBBs1-like sequence (SEQ ID NO: 32) and (SEQ ID NO: 33) are not as potent to UBB as the UBBs1 (SEQ ID NO: 12) siRNA. The siRNA targeting UBBs1-like sequence on UBC (SEQ ID NO: 34) is as potent as the siRNA targeting UBBs1(SEQ ID NO: 12). A UBBs1 scrambled siRNA targeting sequence (SEQ ID NO: 29) does not have a cytotoxic effect and could be a negative control. A new siRNA targeting sequence (SEQ ID NO: 11) is more potent than UBBs1 (SEQ ID NO: 12).

### Example 7: In Vitro Validation of UBB siRNA Library

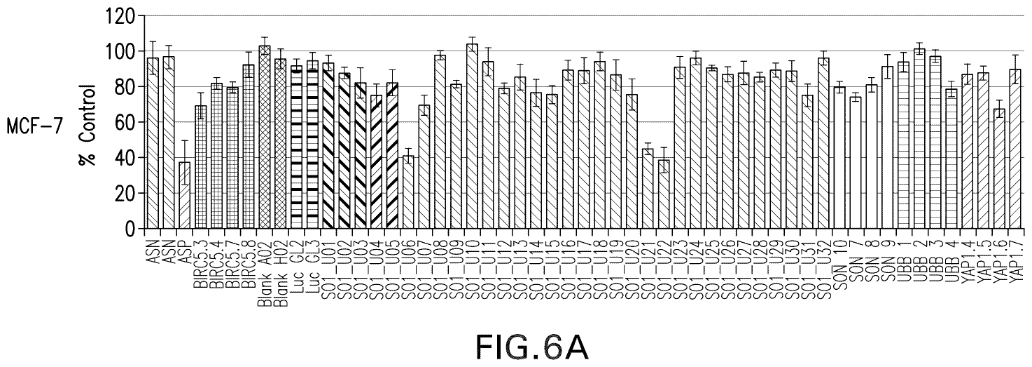

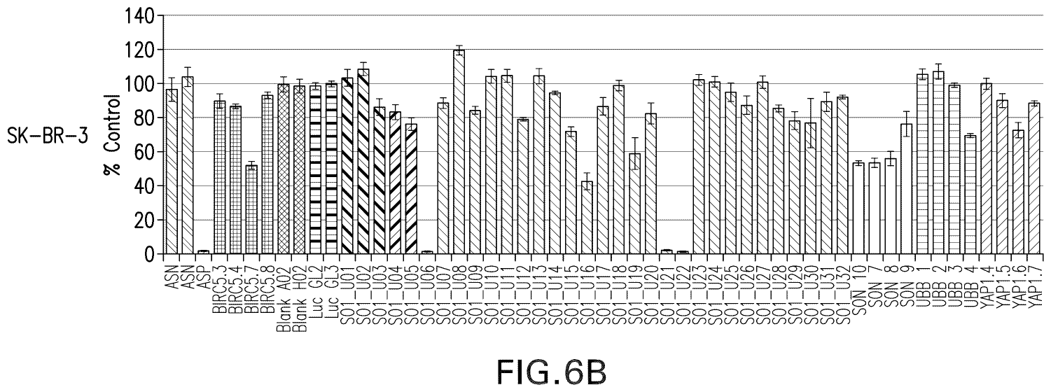

MCF-7 and SK-BR-3 breast cancer cells were treated under standard siRNA transfection conditions with various siRNA compounds including those previously listed as well as controls: ASN siRNA (negative), ASP siRNA (positive) (16.7 nM; 96 hr) (FIG. 6).

The siRNA targeting UBBs1 (SEQ ID NO: 12) is cytotoxic to MCF-7 and SK-BR-3. The siRNA targeting (SEQ ID NO: 34) is as potent as the siRNA to UBBs1(SEQ ID NO: 12) and the siRNA targeting (SEQ ID NO: 11) is more potent than UBBs1 (SEQ ID NO: 12). This experiment demonstrated surprising efficacy of dual UBB and UBC siRNA inhibition on breast cancer cells.

### Example 8: Dose Response of Various siRNA Sequences on Colon Cancer Cells

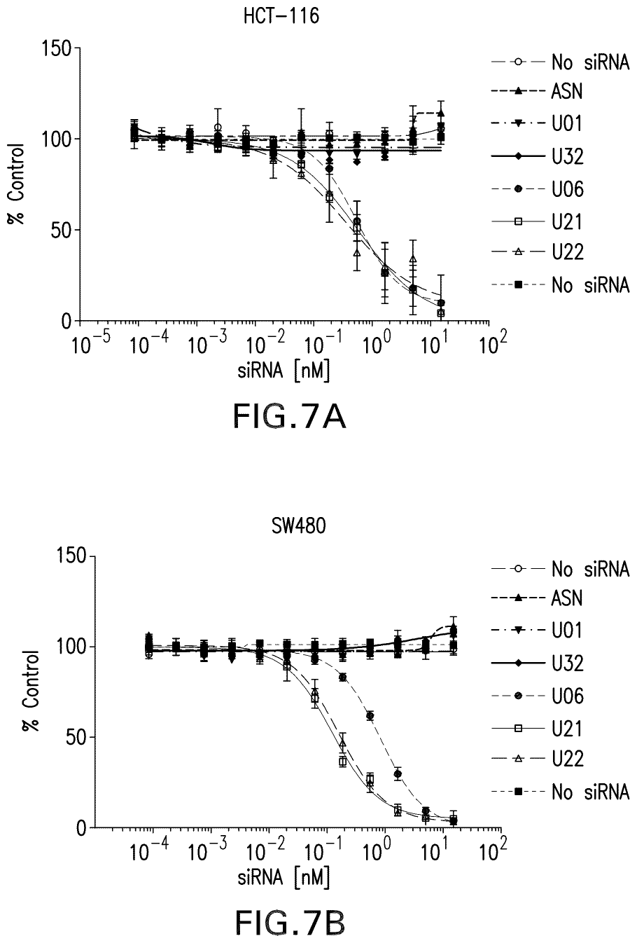

Dose response curve of HCT-116 and various siRNA sequences. Cells were grown to 2,000 cells/well in a 384-well plate, and treated with 62 pM-15 nM of compounds for 96 hours (FIG. 7A).

FIG. 7B: Dose response curve of SW480 and various siRNA sequences. Cells were grown to 2,000 cells/well in a 384-well plate, and treated with 62 pM-15 nM of compounds for 96 hours (FIG. 7B). These results demonstrate that high concentrations of siRNA-aptamer dual targeting chimeras is not necessary to see efficacy in cancer cells.

### Example 9: Silencing of UBB and UBC

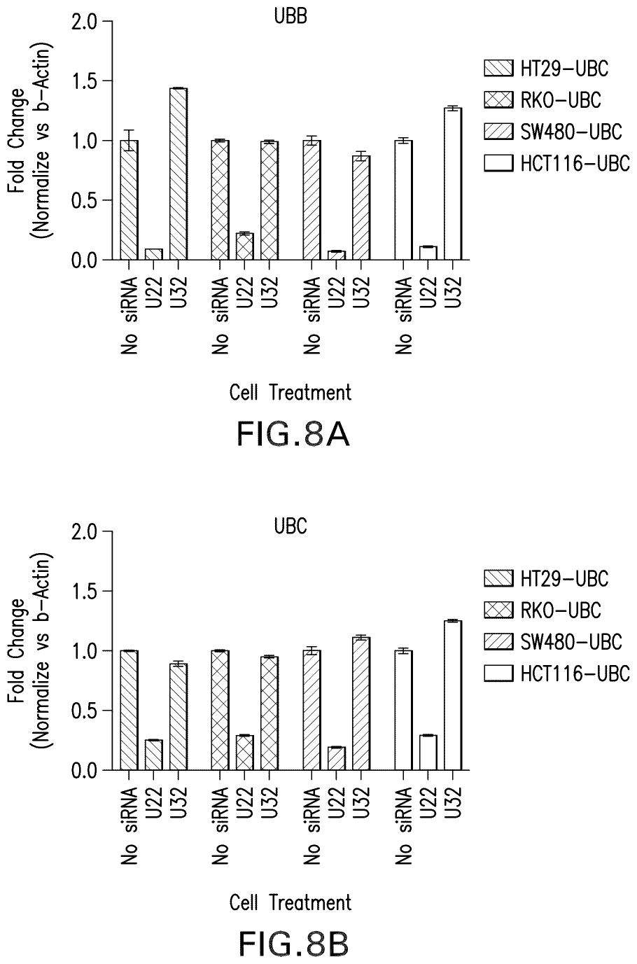

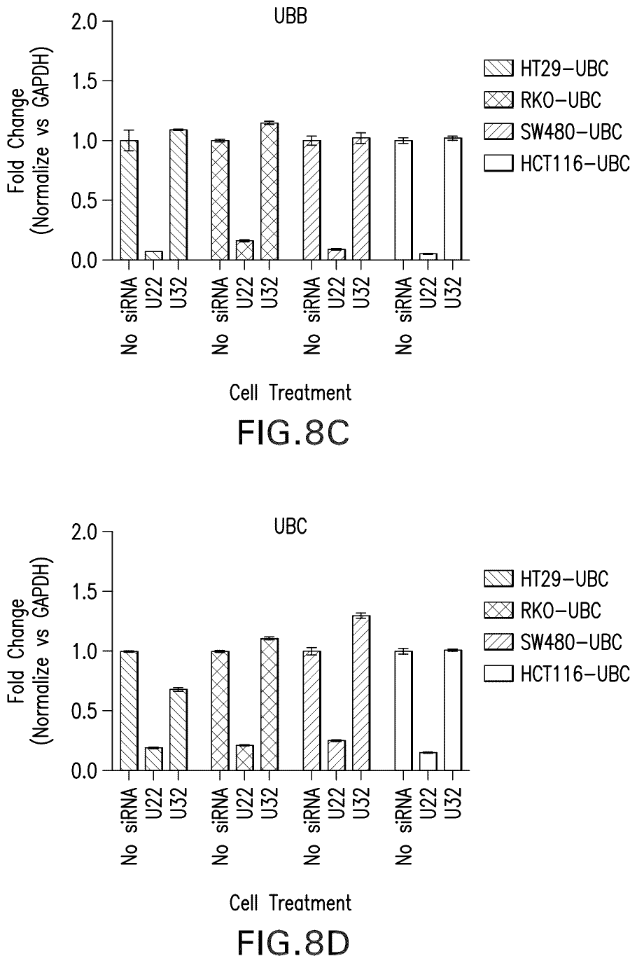

In order to demonstrate that active siRNA targeting (SEQ ID NO: 12) silence both UBB and UBC and other UBB targeting siRNA's do not, a cell assay was performed using HT29, RKO, SW480, and HCT116 cells. Cells were treated with siRNA or control (15 nM siRNA; 20 hr). UBB (FIGS. 8A and 8c) or UBC levels (FIG. 8b or 8d) were measured and normalized by b-Actin (FIGS. 8A and 8B) or GAPDH (FIGS. 8C and 8D).

Results indicate the dual targeting capability of siRNA's to (SEQ ID NO: 12) across multiple cell types.

### Example 10: UBB-UBC Expression in HCT116 Cells Following siRNA Knockdown

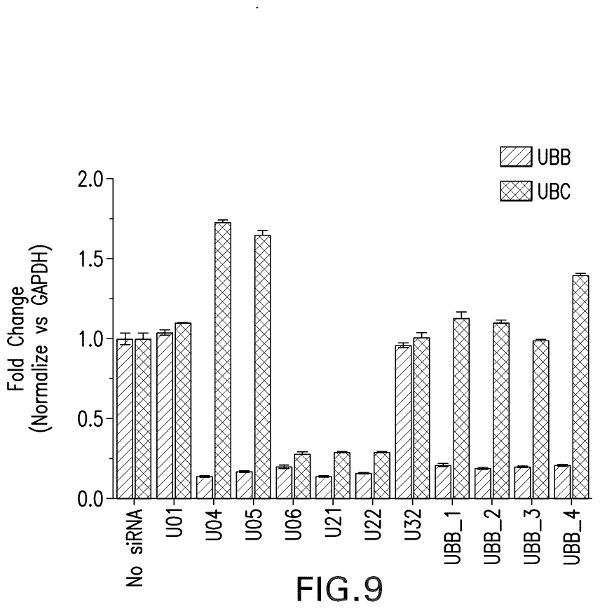

HCT116 cells were treated with the specified siRNA including U01, a Luciferase GL3 siRNA (15 nM siRNA; 20 hr). qPCR results were normalized to GAPDH. Results demonstrate the ability of siRNA's targeting (SEQ ID NO: 69), (SEQ ID NO: 70) and (SEQ ID NO: 71) to dual inhibit UBB and UBC. Control UBB inhibitors are not able to inhibit UBC (FIG. 9).

(SEQ ID NO: 69):
GGCAAAGAUCCAAGAUAAG
(SEQ ID NO: 70):
GGCCAAGAUCCAAGAUAAA
(SEQ ID NO: 71):
AGGCCAAGAUCCAAGAUAA

Additionally, HCT116 cells were treated with another set of UBB/UBC targeted siRNAs.

(SEQ ID NO: 35):
GCCGUACUCUUUCUGACUA (UBB_1G2)
(SEQ ID NO: 36):
GUAUGCAGAUCUUCGUGAA (UBB_2G2)
(SEQ ID NO: 37):
GACCAUCACUCUGGAGGUG (UBB_3G2)
(SEQ ID NO: 38):
CCCAGUGACACCAUCGAAA (UBB_4G2)
(SEQ ID NO: 39):
GUGAAGACCCUGACUGGUA (UBC_1G6)
(SEQ ID NO: 40):
AAGCAAAGAUCCAGGACAA (UBC_2G6)
(SEQ ID NO: 41):
GAAGAUGGACGCACCCUGU (UBC_3G6)
(SEQ ID NO: 42):
GUAAGACCAUCACUCUCGA (UBC_4G6)

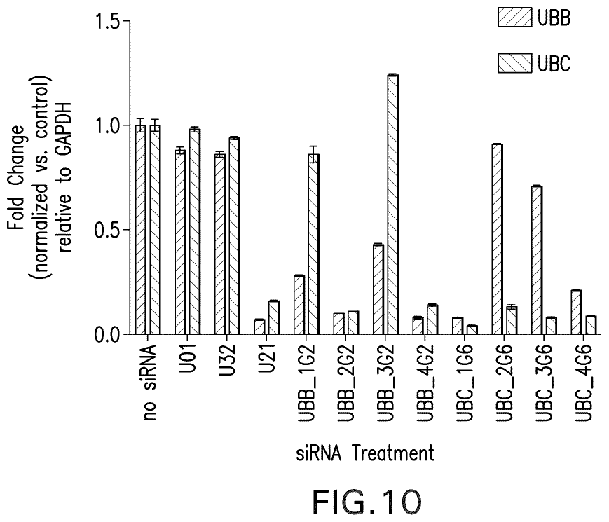

siRNA targeting (SEQ ID NO: 36) and (SEQ ID NO: 38) and (SEQ ID NO: 39) and (SEQ ID NO: 42) demonstrated significantly diminished UBB and UBC expression levels. (FIG. 10)

### Example 11

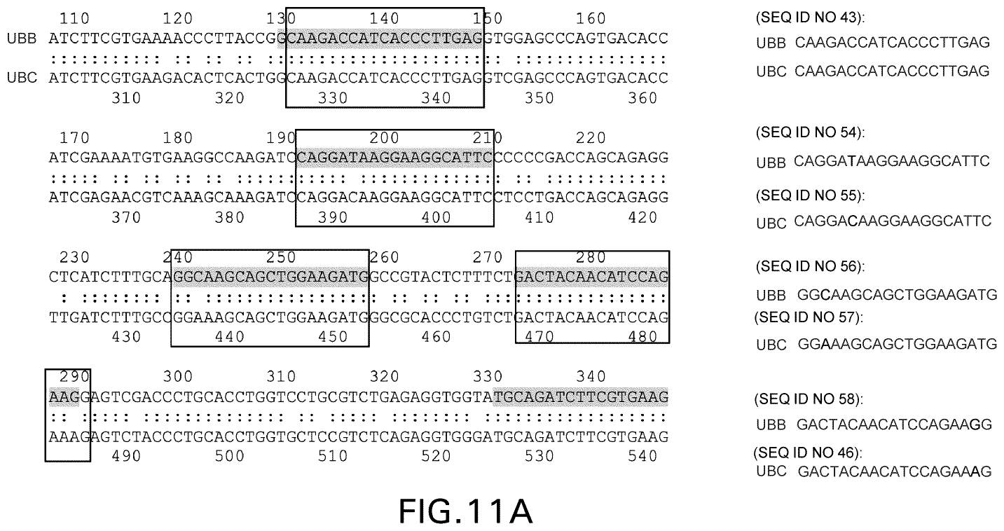

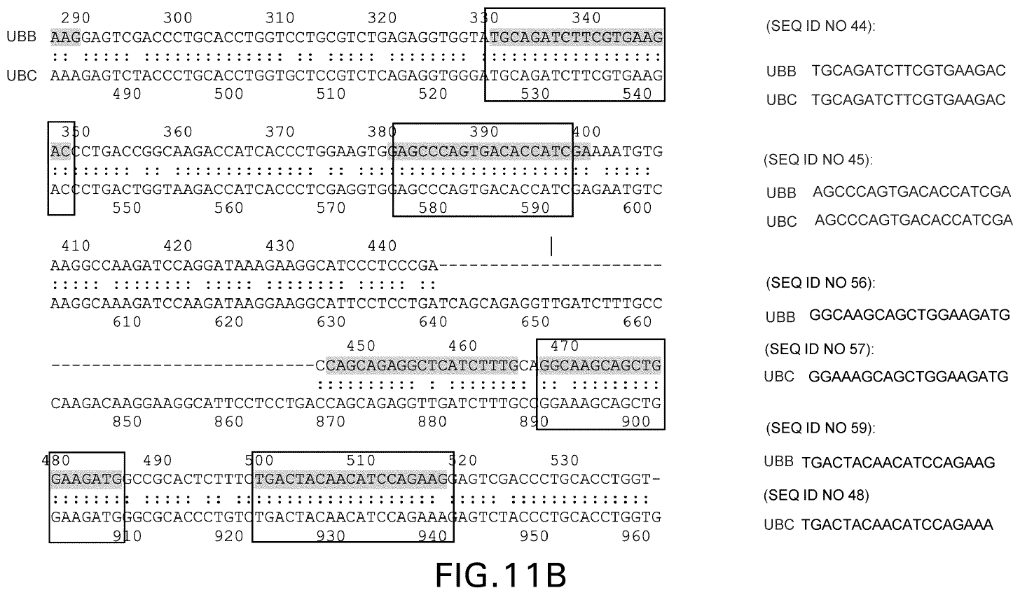

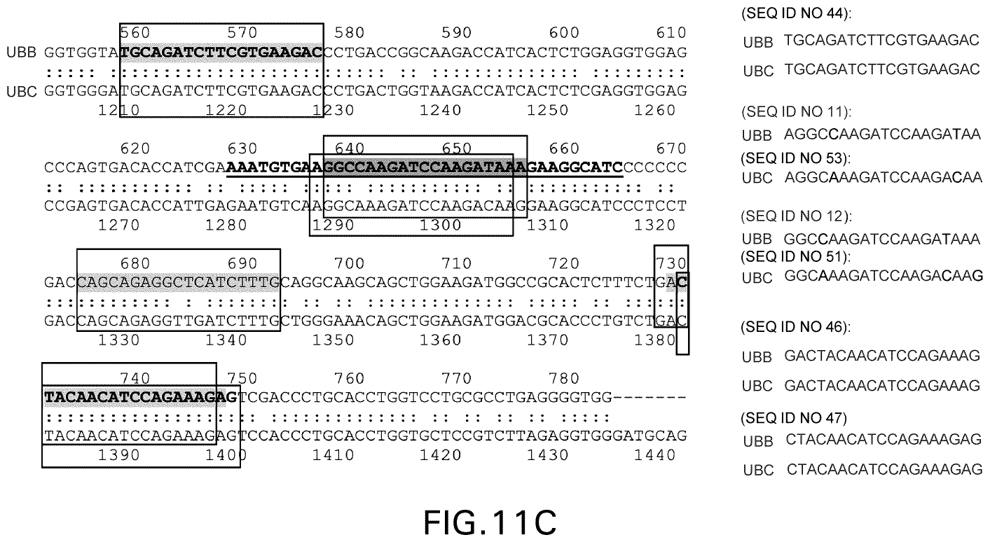

Depicts BLAST results of homologous regions between UBB and UBC mRNA at regions with 19/19, 18/19 and 17/19 identity over the 19 nt stretch (FIG. 11).

Dual UBB and UBC siRNA Targeting Sequences:

(SEQ ID NO: 43):
5′-CAAGACCATCACCCTTGAG-3′
(SEQ ID NO: 44):
5′-TGCAGATCTTCGTGAAGAC-3′
(SEQ ID NO: 45):
5′-AGCCCAGTGACACCATCGA-3′
(SEQ ID NO: 46):
5′-GACTACAACATCCAGAAAG-3′
(SEQ ID NO: 47):
5′-CTACAACATCCAGAAAGAG-3′
(SEQ ID NO: 48):
5′-TGACTACAACATCCAGAAA-3′

UBB Similar to UBC siRNA Targeting Sequences:

(SEQ ID NO: 49):
5′-AGTGACACCATCGAAAATG-3′
(SEQ ID NO: 50):
5′-AGGCAAAGATCCAAGATAA-3′
(SEQ ID NO: 51):
5′-GGCAAAGATCCAAGACAAG-3′
(SEQ ID NO: 52):
5′-CAAGGCAAAGATCCAAGAC-3′
(SEQ ID NO: 53):
5′-AGGCAAAGATCCAAGACAA-3′
(SEQ ID NO: 54):
5′-CAGGATAAGGAAGGCATTC-3′
(SEQ ID NO: 55):
5′-CAGGACAAGGAAGGCATTC-3′
(SEQ ID NO: 56):
5′-GGCAAGCAGCTGGAAGATG-3′
(SEQ ID NO: 57):
5′-GGAAAGCAGCTGGAAGATG-3′
(SEQ ID NO: 58):
5′-GACTACAACATCCAGAAGG-3′
(SEQ ID NO: 59):
5′-TGACTACAACATCCAGAAG-3′

(SEQ ID NO: 42) was identified 2× in UBC and 1× in UBB. (SEQ ID NO: 44) was identified 4× in UBC and 1× in UBB. (SEQ ID NO: 45) was identified 2× in UBC and 3× in UBB. (SEQ ID NO: 46) was identified 7× in UBC and 1× in UBB. (SEQ ID NO: 47) was identified 7× in UBC and 1× in UBB.

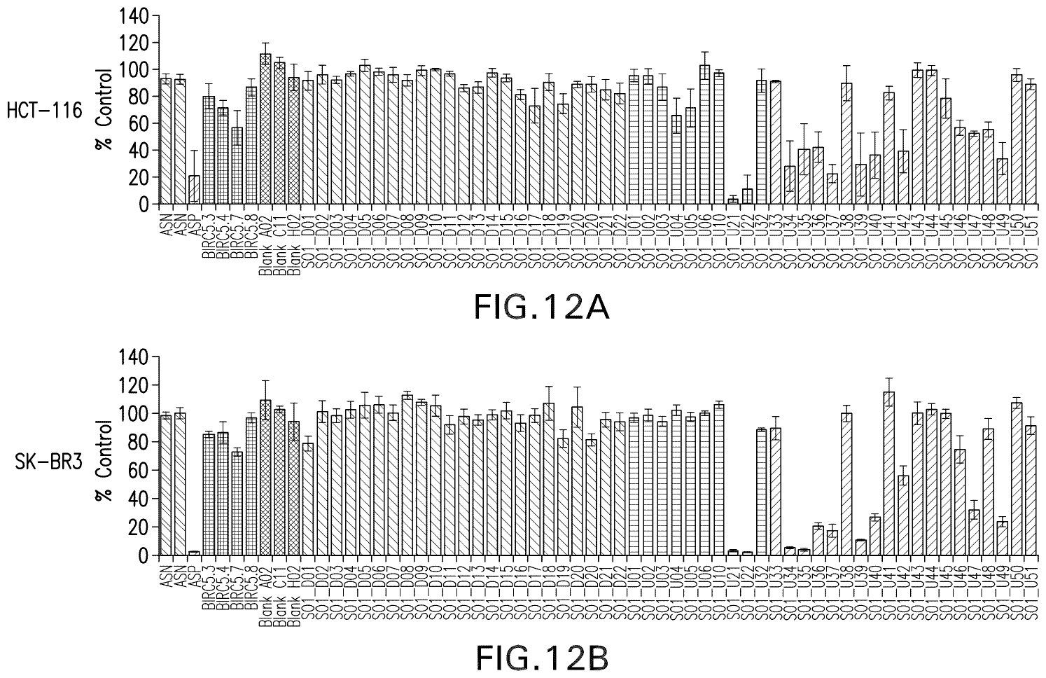

HCT-116 (FIG. 12A), a colon cancer cell line, and SK-BR3 (FIG. 12B), a breast cancer cell line, were treated under standard siRNA transfection conditions with siRNA compounds targeting mRNA sequences previously listed as well as ASN (negative control) and ASP (positive control) (16.7 nM; 96 hr). U32, U50, U51 are negative control siRNAs.

These results identify (SEQ ID NO: 44), (SEQ ID NO: 45), (SEQ ID NO: 46), (SEQ ID NO: 47), and (SEQ ID NO: 49) as siRNA targets with the ability to inhibit both UBB and UBC.

### Example 12

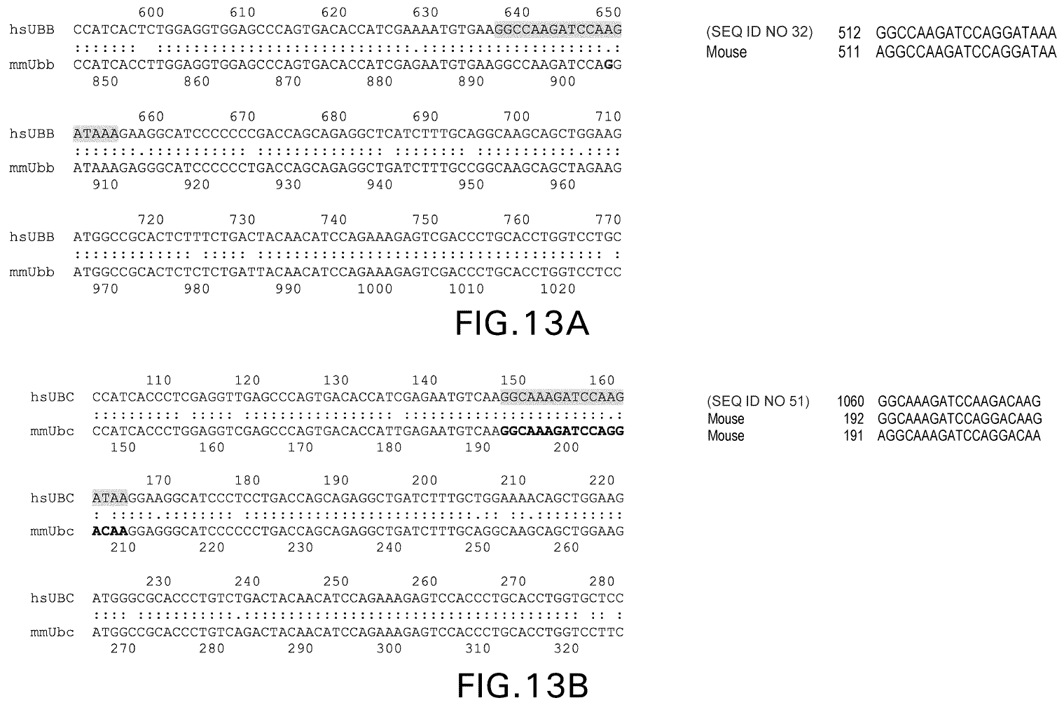

Human UBB and UBC sequences were compared to mouse in order to find homologous regions for in vivo work. (SEQ ID NO: 32) and (SEQ ID NO: 51) sequences were found to be effective siRNA targeting regions for human and contain high homology to mouse. (SEQ ID NO: 32) with minimal nucleotide differences was identified 4× in mouse UBB and (SEQ ID NO: 51) with minimal nucleotide differences was identified 9× in mouse UBC. These sequences will be effective for multi-species in vivo studies (FIG. 13).

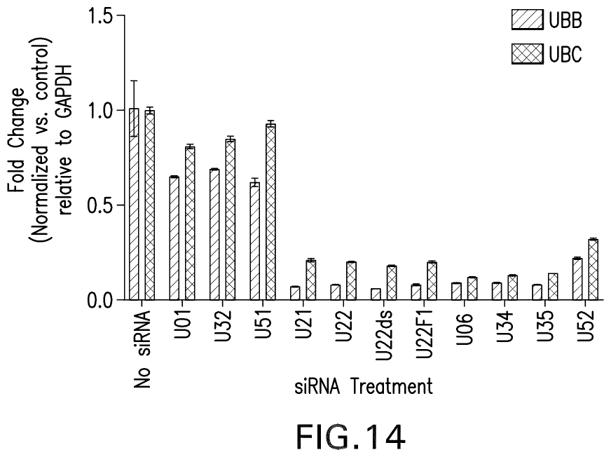

An additional mouse sequence (SEQ ID NO: 60: U52) was tested against UBB and UBC. Additionally, a dicer substrate siRNA (SEQ ID NO: 61: U22ds) and a 2′F pyrimidine modified siRNA (SEQ ID NO: 71: U21F) were included in this experiment. Gene expression of HCT116 cells was measured by qPCR following siRNA treatment and these siRNAs were found to effectively decrease expression of both UBB and UBC (FIG. 14).

(SEQ ID NO: 60):
5′-GGCAAAGAUCCAGGACAAG-3′ (U52)
(SEQ ID NO: 61):
5′-GGCCAAGAUCCAAGAUAAAGAAGGC-3′ (U22ds)

### Example 13: UBB-UBC Modifications

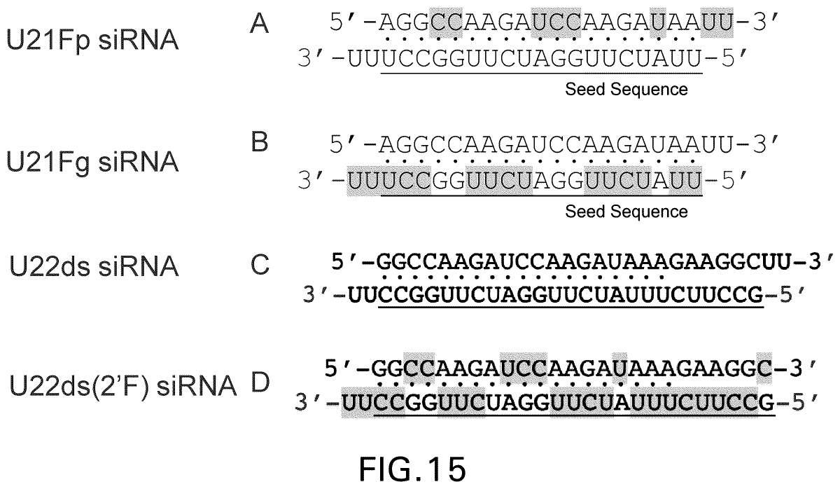

2′F pyrimidine modifications of the siRNA targeting SEQ ID NO: 71 are depicted in FIG. 15. The modifications can either be on the passenger strand, U21Fp (FIG. 15A) or the guide strand, U21Fg (FIG. 15B). The guide strand is underlined.

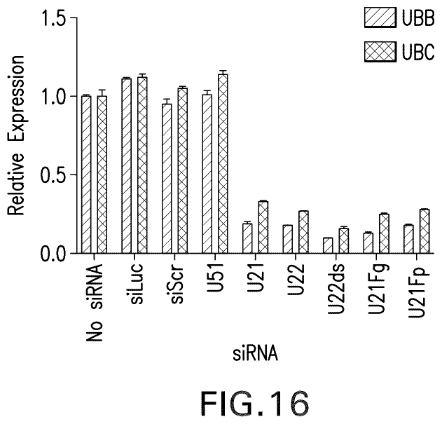

HCT-116 cells were treated with UBB-UBC targeting siRNAs. Modified and unmodified versions of SEQ ID NO: 71 are able to silence UBB and UBC with similar activity to that of unmodified (FIG. 16).

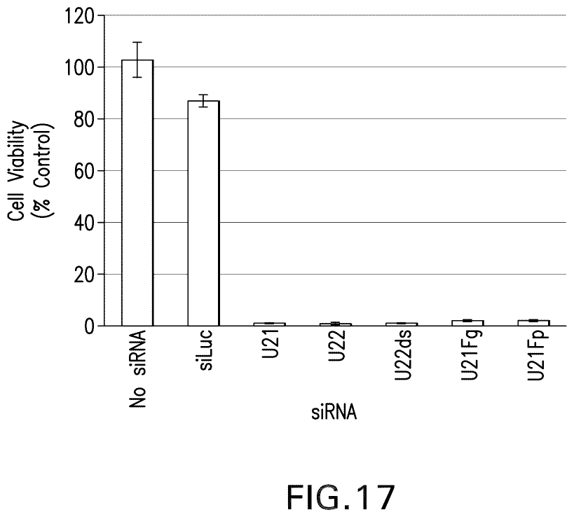

Cell viability was measured, and the silencing of these genes demonstrated >98% cytotoxicity at 96 hours. (FIG. 17)

### Example 14: EPCAM Aptamer Construction

EpCAM aptamers were individually synthesized by in vitro transcription with PCR products as templates. The ssDNA of EpCAM aptamer containing T7 RNA polymerase promoter site (underlined) and adaptor sequence (5′-TAATACGACTCACTATAGCGACTGGTTACCCGGTCGT-3′) (SEQ ID NO: 62) was synthesized from IDT as a PCR template. PCR was performed with forward primer (5′-TAATACGACTCACTATA GCGACTGGTTA-3) (SEQ ID NO: 63) and reverse primer (5′-ACGACCGGGTAACCAGTCGC-3′) (SEQ ID NO: 64). The PCR products were put into T-A cloning pCR 2.1 vector (Invitrogen) and sequenced. Transcription was performed with PCR product as templates using DuraScript transcription kits following manufacture's instruction.

### Example 15: EPCAM—UBB Aptamer-siRNA Chimera Construction

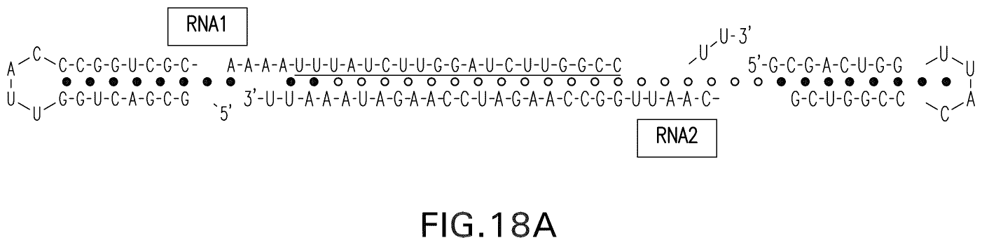

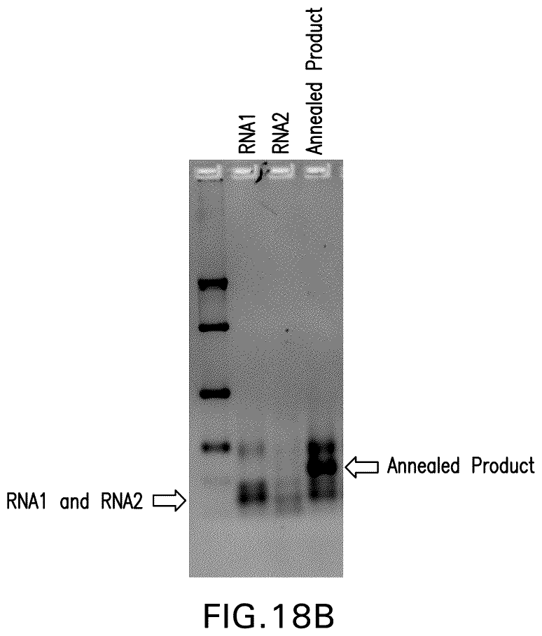

EpCAM-directed aptamers-siRNA chimeras were individually synthesized by in vitro transcription from an annealed DNA templates (FIG. 18A). For RNA 1, two ssDNA containing T7 RNA polymerase promoter site (bolded) and adaptor sequence (5′-GTAATACGACTCACTATAGGCGACTGGTTACCCGGTCGCAATTGGCCAAGATCCAAGATA AATT-3′) (SEQ ID NO: 65) and (5′-AATTTATCTTGGAUCTTGGCCAATTGCGACCGGGTAACCAGTCGCCTATAGTGAGTCGTAT TAC-3′) (SEQ ID NO: 66) were synthesized by IDT as a T7 template. For RNA 2, two ssDNA containing T7 RNA polymerase promoter site (bolded) and adaptor sequence (5′-GTAATACGACTCACTATAGGCGACTGGTTACCCGGTCGCAAAATTTATCTTGGATOTTGGC CTT-3′) (SEQ ID NO: 67) and (5′-AAGGCCAAGATCCAAGATAAATTTTGCGACCGGGTAACCAGTCGCCTATAGTGAGTCGTAT TAC-3′) (SEQ ID NO: 68) were synthesized by IDT as a T7 template. The annealed double stranded DNA for each RNA1 and RNA2 were used as templates for T7 polymerase using DuraScript transcription kits following manufacture's instruction. The two RNAs were further purified and mixed at molar ratio 1:1 and annealed to form the chimeric molecule by heating at 94° C. for 3 min followed by slowly cooling to room temperature within 1 h. Resulting products were run on a gel for confirmation (FIG. 18B)

### Example 16: Conjugating Antibody-siRNA Via Ionic Interactions

Fusion of an antibody with a multi-cationic moiety such as protamine or polyarginine is created when the negative charge of the oligonucleotide backbone and positive charge of the protamine binds the oligonucleotide and protein strongly via ionic interactions. Anti-EpCAM Fab fragment is expressed and purified E104 E. coli cells and is fused at its C terminus to protamine. siRNA is mixed with protamine, anti-EpCAM scFv with protamine, anti-EpCAM Fab fragment with protamine, anti-EpCAM scFv, anti-EpCAM Fab fragment, or PBS at a molar ratio of 6:1 (siRNA concentration, 300 nM) in PBS for 30 min at 4° C.

HT29, SW480, and HCT116 cells are treated at ˜75% confluency in 800 μl in 6-well plates. For controls, cells are transfected with oligofectamine (Invitrogen) or TransIT-siQUEST (Mirus) following the manufacturers' protocol. Cells are analyzed for gene expression and proliferation two days after siRNA treatment.

### Example 17: Conjugating Antibody-siRNA Via Avidin-Based Conjugation

Avidin-based conjugation has greater in vivo stability. Biotinylated siRNA is created with a tetra-ethyleneglycol spacer placed between the 3′-terminus and the biotin group. EpCAM antibodies are purified by protein G affinity chromatography from isolated mouse tissue. Recombinant streptavidin is conjugated to the EpCAMAb with a stable thio-ether linkage in a 1:1 molar ratio, and purified with gel filtration chromatography. The biotinylated siRNA is added to HT29, SW480, and HCT116 cells treated at ˜75% confluency in 800 μl in 6-well plates, 0 to 115 nM siRNA, and 0 to 400 nM EpCAMAb/Streptavidin, unconjugated Streptavidin, unconjugated EpCAMAb, or avidin.

### Example 18: Conjugating Antibody-siRNA Via Direct Conjugation

Direct conjugation allows the use of the same linkers found in antibody-drug-conjugates such as cleavable, disulfide, and non-cleavable linkers. The linker in the siRNA is usually incorporated into the sense strand rather than the anti-sense strand. 3′ amine modified siRNA is reacted with a reducible N-succinimidyl-4-(2-pyridyldithio)butyrate (SPDB) or non-reducible succinimidyl-4-(N-maleimidomethyl]cyclohexane-1-carboxylate) (SMCC) NHS (N-hydroxysuccinimide) linker to form a thiol-reactive siRNA-linker adduct, and this adduct is then reacted with thiol groups on an engineered anti-EpCAM antibody in which a cysteine residue had been introduced in the heavy chain, to covalently link the siRNA via a thio-ester bond. Alternatively, azide-alkyne click chemistry, transglutaminase-click chemistry, beta-lactam conjugation, or conjugation with a disuccinimidyl linker will result in directly conjugated antibody-siRNA.

### Example 19. In Vivo Inhibition of UBB and UBC mRNA by the UBB-UBC Dual Targeting siRNA

Male NSG mice are injected subcutaneously (HCT116) or intrasplenically (mHCT116) with human HCT116 CRC tumor cells to disseminate LM, whereas experimental controls receive saline. Huot et al. demonstrated elevated ubiquitin expression in this model (Huot et al., Dis Models & Mech, 13:1754-8403 (2020)).

Mice will be treated with the dual UBB-UBC targeting siRNAs conjugated to EPCAM aptamer, EpCAM-scrambled siRNA, or vehicle by intraperitoneal injection of 0.1 ml of the indicated solution. Mice will be treated with a dose of dual targeting siRNA sufficient to inhibit expression of UBB and UBC by at least 10%, at least 15%, at least 20%, at least 25%, at least 30%, at least 40%, at least 50%, at least 60%, at least 70%, at least 80% or at least 90% or more, for at least 5, more preferably 7, 10, 14, or 18 days. Alternatively, mice will be dosed multiple times in order to inhibit expression of UBB and UBC by at least 10%, at least 15%, at least 20%, at least 25%, at least 30%, at least 40%, at least 50%, at least 60%, at least 70%, at least 80% or at least 90% or more, for at least 5, more preferably 7, 10, 14, or 18 days. All the mice are sacrificed on day 18, and tumors are collected for quantitation.

### Example 20: Lipid Bioconjugate Addition

Lipid conjugation prolongs circulation time, stability, and bioavailability of siRNAs in vivo.

Cholesterol is a well-known conjugate enabling efficient cellular and tissue delivery following direct oligonucleotide conjugation. Cholesterol-siRNA conjugation is standard practice and can be done following standard protocols by RXi Pharmaceuticals, Arrowhead Pharmaceutical, or Nature Biotechnology 36:1164-1173 (2018). Alternatively, siRNAs are conjugated to lithocholic acid, a-Tocopherol, myristic acid, Docosanoic acid, or Docosahexaenoic acid.

Altering the nature of the lipid conjugate profoundly affects tissue distribution, therefore the most effective lipids for each drug of interest will need to be identified. Male NSG mice are injected subcutaneously (HCT116) or intrasplenically (mHCT116) with human HCT116 CRC tumor cells to disseminate LM. Mice will be treated with the dual UBB-UBC targeting siRNAs conjugated on one end to lithocholic acid, a-Tocopherol, Docosanoic acid, or Docosahexaenoic acid and the other end with EPCAM aptamer. Controls will include scrambled siRNA or vehicle. Construct will be delivery by intraperitoneal injection of 0.1 ml of the indicated solution. ng of siRNA/mg of tissue will be measured for fallopian tube, bladder, adrenal glands, skin, spleen, pancreas, heart, intestine, ling, thymus, muscle, fat, and tumor. Additionally, mRNA expression will be measured to demonstrate efficacy of inhibition two days after administration.

### Example 21: In Vivo Impact of UBB and UBC mRNA Inhibition on Tumor Size

To assess the impact of a compound comprising dual targeting siRNA conjugated to EPCAM aptamer on tumor growth in vivo, subcutaneous HCT-116 xenografts will be established in athymic nu/nu male mice. The compound will be injected intraperitoneally to tumor-bearing mice every other day for 1 week and every day for the following two weeks. Control mice will be injected intraperitoneally with equivalent volume of PBS or EpCAM—scrambled siRNA. All the mice are sacrificed on day 21, and tumors are collected for quantitation.

In certain embodiments, the invention provides pharmaceutical compositions containing a dual targeting siRNA agent, as described herein, and a pharmaceutically acceptable carrier.

The pharmaceutical compositions featured herein are administered in dosages sufficient to inhibit expression of the target genes. In general, a suitable dose of siRNA will be in the range of 0.01 to 200.0 milligrams per kilogram body weight of the recipient per day, generally in the range of 1 to 50 mg per kilogram body weight per day.

The pharmaceutical composition may be administered once daily, or the siRNA may be administered as two, three, or more sub-doses at appropriate intervals throughout the day or even using continuous infusion or delivery through a controlled release formulation. In that case, the siRNA contained in each sub-dose must be correspondingly smaller in order to achieve the total daily dosage. The dosage unit can also be compounded for delivery over several days, e.g., using a conventional sustained release formulation which provides sustained release of the siRNA over a several day period. Sustained release formulations are well known in the art and are particularly useful for delivery of agents at a particular site, such as could be used with the agents of the present invention. In this embodiment, the dosage unit contains a corresponding multiple of the daily dose.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A therapeutic composition comprising a ligand and a cytotoxin, wherein the cytotoxin is a siRNA construct.

2. The composition of claim 1, wherein the ligand comprises a monoclonal antibody, antibody mimics, antibody fragment, a centyrin, a cytokine, a growth factor, nucleic acid, nanoparticle, polymer, protein, aptamer, lipid, small molecule, radionucleotide, peptide, or a peptide fragment.

3. The composition of claim 2, wherein the ligand comprises Dextran cage, nanotube, quantum dot, magnetic nanoparticles, HPMA-s-APMA, PNIPAAm, PEG, Penatratin, Transportan, Tat, Anandamine, DAC, EPA, PC-DCA, a-tocopherol, cholesterol, GalNac, Lac, M6P, DUPA, Folate, CpG1668, LGRH peptide, cRGD, Tat-AHNP, Octreotide, IGF1 mimetic peptide, IL2, anti-CD22 dsFv, anti-CD25 scFv, GMCSF, Anti-CD-25 mAb, Anti-CD3 biFv, Anti-CD22 Fab, Anti-CD30 mAb, Anti-CD33 mAb, Variant IL3, Mesolthelin, cholesterol, EpCAM, EGFRvIII, EGFR, ErbB2, IL13R, IL4R, or TfR.

4. A therapeutic composition comprising a ligand that specifically binds at least one target protein and a cytotoxin that is processed by cellular RNAi machinery to produce one or more siRNAs wherein at least one of said siRNAs specifically inhibits the expression of one or more different genes.

5. The composition of claim 4, wherein at least one of said siRNAs inhibits the expression of UBB and UBC.

6. A therapeutic construct comprising a ligand that specifically binds at least one target protein and a cytotoxin that is processed by cellular RNAi machinery to produce one or more siRNAs wherein at least one of said siRNAs specifically inhibits the expression of two or more different genes.

7. The construct according to claim 6 wherein the different genes comprise UBB and also UBC.

8. The construct according to claim 6 wherein the construct comprises the siRNA sequence SEQ ID NO: 36, SEQ ID NO: 38, SEQ ID NO: 40, SEQ ID NO: 42, SEQ ID NO: 60, SEQ ID NO: 61, SEQ ID NO: 70, SEQ ID NO: 71 or SEQ ID NO: 72.

9. A composition or construct according to claim 1 wherein the ligand demonstrates cancer cell tropism.

10. A therapeutic composition comprising a ligand and a cytotoxin, wherein the cytotoxin is a UBB/UBC inhibitor.

11. The composition of claim 10 wherein the UBB/UBC inhibitor is selected from the group consisting of siRNA, mRNA, small molecule, antibody, aptamer, and antisense oligonucleotide.

12. The composition of claim 11 wherein the UBB/UBC inhibitor is siRNA.

13. The composition of claim 12 wherein the UBB/UBC inhibitor comprises a siRNA sequence selected from the group consisting of SEQ ID NO: 36, SEQ ID NO: 38, SEQ ID NO: 40, SEQ ID NO: 42, SEQ ID NO: 60, SEQ ID NO: 61, SEQ ID NO: 70, SEQ ID NO: 71 and SEQ ID NO: 72.

14. The composition of claim 13 wherein the ligand is conjugated to the UBB/UBC inhibitor and wherein the ligand specifically binds a cell surface protein expressed by a tumor.

15. The composition or construct according to claim 1 wherein the ligand binds to ERBB2, ERBB3, FOLH1, CD44, EPCAM, FOLH1, PSCA, PDCD1, TACSTD2, NT5E, PDCD1, CTLA4, LAG3 or HAVCR2.

16. A method for treating cancer comprising administering a cytotoxin comprising an siRNA.

17. The method of claim 16 wherein the cytotoxin inhibits the expression of UBB and UBC.

18. A method for treating cancer comprising administering a cytotoxin comprising a dual, single molecule UBB/UBC inhibitor.
